(19) United States
(12) Patent Application Publication
(10) Pub. No.: US 2023/0374092 A1
(43) Pub. Date: Nov. 23, 2023

(54) METHODS OF TREATING NEURONAL DISEASES USING AIMP2-DX2 AND OPTIONALLY A TARGET SEQUENCE FOR miR-142 AND COMPOSITIONS THEREOF

(71) Applicant: Generoath Co., Ltd., Seoul (KR)

(72) Inventors: Jin Woo CHOI, Seoul (KR); Kyunghwa BAEK, Gangneung-si, Gangwon-do (KR)

(21) Appl. No.: 18/246,575

(22) PCT Filed: Sep. 30, 2021
(86) PCT No.: PCT/IB2021/059017

§ 371 Date: Mar. 24, 2023

Related U.S. Application Data
(60) Provisional application No. 63/085,950, filed on Sep. 30, 2020.

(51) Int. Cl. C07K 14/47; C12N 15/86; A61P 25/16; A61P 25/28; C12N 15/113

(52) U.S. Cl. CPC C07K 14/4747; C12N 15/86; A61P 25/16; A61P 25/28; C12N 15/113; A61K 48/00

(57) ABSTRACT

Disclosed herein are methods of treating neuronal diseases, comprising administering to a subject in need thereof a vector comprising AIMP2-DX2 and optionally a target sequence for miR-142.

### CROSS-REFERENCE TO RELATED APPLICATIONS

This application claims benefit of the filing date of U.S. application Ser. No. 63/085,950, filed Sep. 30, 2020, the disclosure of which is incorporated herein by reference in its entirety.

### REFERENCE TO SEQUENCE LISTING SUBMITTED ELECTRONICALLY

The content of the electronically submitted sequence listing in ASCII text file (Name: 2493-0004WO01_Sequence Listing_ST25.txt; Size: 28 KB; and Date of Creation: Sept. 30, 2021) filed with the application is incorporated herein by reference in its entirety.

### FIELD OF THE INVENTION

Disclosed herein are methods of treating neuronal diseases, comprising administering to a subject in need thereof a vector comprising AIMP2-DX2 and optionally a target sequence for miR-142.

### BACKGROUND OF THE INVENTION

The brain of mammals can execute complex functions through establishment of systemic neural network after having undergone a series of processes including division, differentiation, survival and death of neuronal stem cells, and formation of synapses, etc. Neurons in the animal brain continuously produce a wide range of substances necessary in the growth of nerves even during their matured state, thereby inducing the growths of axon and dendrite. Moreover, it can be said that they continuously undergo differentiation since there is ceaseless synaptic remodeling of the neural network and synaptic connections whenever new learning and memorization is executed. Neurons undergo apoptosis if they are unable to receive target-derived survival factors such as neural growth factor in the process of cell differentiation and synaptic formation and apoptosis due to stress and cytotoxic agents become the main cause of degenerative cerebral disorders. When the peripheral nervous system of animals, unlike the central nervous system, is damaged, axons are regenerated over prolonged period of time. Axons at the back of the damaged nerves are degenerated by the process known as Wallerian degeneration and the cell body of the nerve recommences axonal regrowth while the Schwann cells are regenerated after having undergone a regeneration process, including determination of the target nerve through survival and extinction following division prior to undergoing differentiation, etc. again.

Throughout the world, there is a trend of continued increase in manifestation of neurodegenerative diseases every year along with the rapid increase in aged population. As the definitive prevention and treatment methods have not been discovered yet, there still is no drug with outstanding efficacy in treating such diseases. In addition, existing drugs and therapies used for these disorders frequently display side effects and toxicity arising from prolonged administration. Moreover, since they only have the effect of temporarily reducing the extent of symptoms or delaying the progress of the symptoms rather than complete treatment of the diseases, it is urgent to excavate and develop materials with decisive treatment efforts while reducing side effects and toxicity.

Approximately 600 cases of clinical trials on gene therapy on human subjects have been executed and were in progress until 2002 since the commencement of clinical trials in 1990 for the first time. On the foundation of the completion of human genome sequence analysis in 2003, development of new gene therapies will accelerate in the future through excavation of a diverse range of genes. However, 75% of the gene therapies that have been approved until now are targeted at monogenic diseases such as cancer or cystic fibrosis, etc., and there is no active development of gene therapy drugs for neural disorders or regeneration (Recombinant DNA consultation paper of NIH, USA (2002); Gene Therapy Clinical Trials, J. Gene Med. (2002) www.wiley.co.uk/genmed). Nonetheless, development of gene therapy by using neural growth factor such as NT-3 and glial derived neuronal factor (GDNF) for the treatment and regeneration of sensory neurons for Parkinson's disease is being attempted already (GDNF family ligands activate multiple events during axonal growth in mature sensory neurons (Mol. Cell. Neurosci. 25:4453-4459 (2004)). Since there is sluggish progress in the overall neuroscience researches on the cerebral functions in relation to disorders of nerve system, development of treatment drugs for various chronic disorders of nervous system is also confronted with difficulties.

AIMP2-DX2 is an alternative, antagonistic splicing variant of AIMP2, which is a multifactorial apoptotic gene. AIMP2-DX2 is known to suppress cell apoptosis by hindering the functions of AIMP2. AIMP2-DX2, acting as competitive inhibitor of AIMP2, suppresses TNF-alpha mediated apoptosis through inhibition of ubiquitination/degradation of TRAF2. In addition, it had been reported that AIMP2-DX2 has been confirmed as an existing lung cancer induction factor and, in the existing research, it was confirmed that AIMP2-DX2, manifested extensively in cancer cells, induces cancer by interfering with the cancer suppression functions of AIMP2. Moreover, it was discovered that manifestation of AIMP2-DX2 in normal cell progresses cancerization of cells while suppression of manifestation of AIMP2-DX2, suppresses cancer growth, thereby displaying treatment effects.

It has also been determined that AIMP2-DX2 can be useful in treating neuronal diseases (KR10-2015-0140723 (2017) and US2019/0298858 (pub. Oct. 23, 2019).

### SUMMARY OF THE INVENTION

Disclosed herein are methods for delaying disease onset of a subject with amyotrophic lateral sclerosis (ALS), comprising administering to the subject a recombinant vector comprising an exon 2-deleted AIMP2 variant (AIMP2-DX2) gene.

Disclosed herein are methods of inhibiting neuronal cell death in a subject with amyotrophic lateral sclerosis (ALS), comprising administering to the subject a recombinant vector comprising an exon 2-deleted AIMP2 variant (AIMP2-DX2) gene.

Disclosed herein are methods of treating muscle atrophy in a subject in need thereof, comprising administering to the subject a recombinant vector comprising an exon 2-deleted AIMP2 variant (AIMP2-DX2) gene. In some embodiments, the subject has amyotrophic lateral sclerosis (ALS). In some embodiments, the subject has spinal muscular atrophy (SMA).

Disclosed herein are methods for increasing survival rate or prolonging lifespan of a subject with Parkinson's disease (PD), comprising administering to the subject a recombinant vector comprising an exon 2-deleted AIMP2 variant (AIMP2-DX2) gene.

Disclosed herein are methods of preventing behavior deficit, restoring a motor symptom, and/or reducing neuronal damage in a subject with Parkinson's disease (PD), comprising administering to the subject a recombinant vector comprising an exon 2-deleted AIMP2 variant (AIMP2-DX2) gene.

Disclosed herein are methods of inhibiting amyloid beta oligomer (Aβ-O)-induced neuronal cell death or Aβ-O-induced p53 expression in a subject with Alzheimer's disease (AD), comprising administering to the subject a recombinant vector comprising an exon 2-deleted AIMP2 variant (AIMP2-DX2) gene.

Disclosed herein are methods of inhibiting neuromuscular junction (NMJ) damage in a subject with spinal muscular atrophy (SMA), comprising administering to the subject a recombinant vector comprising an exon 2-deleted AIMP2 variant (AIMP2-DX2) gene.

Disclosed herein are methods of inhibiting neuromuscular junction (NMJ) damage, inhibiting NMJ block induced respiratory failure, difficulty in breathing, inhibiting NMJ block induced muscle twitching or fasciculation, in a subject with amyotrophic lateral sclerosis (ALS), comprising administering to the subject a recombinant vector comprising an exon 2-deleted AIMP2 variant (AIMP2-DX2) gene.

Disclosed herein are methods of suppressing anoikis, and/or increasing laminin receptor stabilization in a subject with amyotrophic lateral sclerosis (ALS), Parkinson's disease (PD), comprising administering to the subject a recombinant vector comprising an exon 2-deleted AIMP2 variant (AIMP2-DX2) gene.

The recombinant vector can further comprise an miR-142 target sequence.

The vector can further comprise a promoter operably linked to the AIMP2-DX2. In some embodiments, the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter or opsin promoter.

The miR-142 target sequence can be 3′ to the AIMP2-DX2 gene.

In some embodiments, the AIMP2-DX2 gene comprises a nucleotide sequence encoding an amino acid sequence that is at least 90% identical to SEQ ID NO:2, 13, 14, 15, 16, 17, 18, 19, or 20.

In some embodiments, the AIMP2-DX2 gene comprises a nucleotide sequence encoding an amino acid sequence of SEQ ID NO:2, 13, 14, 15, 16, 17, 18, 19, or 20.

In some embodiments, the AIMP2-DX2 gene does not have an exon comprising a nucleotide sequence encoding an amino acid sequence that is at least 90% identical to SEQ ID NO:10 or 11.

In some embodiments, the AIMP2-DX2 gene does not have an exon comprising a nucleotide sequence encoding an amino acid sequence of SEQ ID NO:10 or 11.

The miR-142 target sequence can comprise a nucleotide sequence comprising ACACTA. In some embodiments, the miR-142 target sequence comprises ACACTA and 1-17 additional contiguous nucleotides of SEQ ID NO:5. In some embodiments, the miR-142 target sequence comprises a nucleotide sequence at least 50% identical to a nucleotide sequence of SEQ ID NO:5 (TCCATAAAGTAGGAAACACTACA). In some embodiments, the miR-142 target sequence can comprise a nucleotide sequence of SEQ ID NO:5.

In some embodiments, the miR-142 target sequence comprises ACTTTA. In some embodiments, the miR-142 target sequence comprises ACTTTA and 1-15 additional contiguous nucleotides of SEQ ID NO:7. In some embodiments, the miR-142 target sequence comprises a nucleotide sequence at least 50% identical to a nucleotide sequence of SEQ ID NO:7 (AGTAGTGCTTTCTACTTTATG). In some embodiments, the miR-142 target sequence comprises a nucleotide sequence of SEQ ID NO:7.

The miR-142 target sequence can be repeated 2-10 times in the vector disclosed herein.

The vector can be a viral vector. The viral vector can be an adenovirus, adeno-associated virus, lentivirus, retrovirus, human immunodeficiency virus (HIV), murine leukemia virus (MLU), avian sarcoma/leukosis (ASLV), spleen necrosis virus (SNV), Rous sarcoma virus (RSV), mouse mammary tumor virus (MMTV), Herpes simplex virus, or vaccinia virus vector.

### BRIEF DESCRIPTION OF THE FIGURES

FIG. 1 illustrates an example recombinant vector.

FIG. 2 shows the nerve cell-specific expression effect of a recombinant vector under an in vitro environment.

FIG. 3 shows brain specific expression following intraparenchymal (substantia nigra) injection of scAAV-DX2-miR142-3pT in a Parkinson's Disease model.

FIG. 4 shows an miR142-3pT (target) sequence (SEQ ID NO:6) with 4 repeats of miR142-3pT (underlined).

FIG. 5A shows a schematic of miR142-3pT with 1×, 2×, and 3× repeats, and mutant sequence. FIG. 5B shows miR142-3p inhibition on DX2 expression with 1×, 2×, and 3× repeats of miR-142-3pT.

FIG. 6 shows that a core binding sequence is important in DX2 inhibition. A vector with Tseq ×3 repeats, which showed significant inhibition of DX2 (FIG. 5B), and DX2 construct were used as controls. 100 pmol of miR-142-3p treatment inhibited Tseq ×3 vector significantly but DX2 and mutant sequence were not inhibited.

FIG. 7 shows total RNA extracted from the spinal cord of ALS model following intrathecal injection of scAAV2-DX2-miR142-3p. qRT-PCR was performed.

FIG. 8 shows nerve cell-specific expression effect of an expression vector of the invention under an in vitro environment.

FIGS. 9A-9E. DX2 transgenic mice recover motor symptoms in rotenone-treated mice. FIG. 9A. TH expression was analyzed with mice brain in the indicated mice. The black dotted square shows TF-stained regions. FIG. 9B. Rotarod analysis. Latency to fall in rotenone-treated wild type and DX2 transgenic (TG) mice. FIG. 9C. The Pole test. Vertical movement (left panel) and T-turn time (right panel) in rotenone-treated wild type and DX2 TG mice. Animals; n=6 (in each group), ns; non-significant, **P<0.01, *P<0.05, t-test. FIGS. 9D and 9E. DX2 improves neuronal damage and behavior in rotenone-induced PD mouse model. FIG. 9D. The pole test. scAAV-DX2 recovered motor coordination and balance in the rotenone-treated PD mouse model. “Con” and “GFP” indicate wild type and rotenone-treated GFP injection mice. “Dose 1” and “Dose 2” represent the different injection dose of DX2 in rotenone-treated mice. FIG. 9E. Immunohistochemistry and immunofluorescence image of the mouse substantia nigra. The upper panel shows TH-positive cells in the striatum and the lower panel indicates the distribution of an injected-GFP expressing virus. The black dotted square indicates the stained region of TH. Animals; n=5 (in each group), ns; non-significant, **P<0.05, *P<0.01, t-test.

FIGS. 10A-10H. DX2 prevents behavioral deficits in the 6-OHDA-induced PD model. FIG. 10A. scAAV-DX2-treated mouse showed lower levels of contralateral rotation compared to that of saline or vehicle (GFP), indicating that DX2 attenuated damage in dopaminergic neurons. FIG. 10B. DX2-treated mice showed increased contralateral forepaw contacts, indicating that AAV-DX2 attenuated unilateral damage in dopaminergic neurons. FIG. 10C. AAV-DX2 treated mouse showed less right-biased body swing. Animals; saline (saline-treated wild type mice) n=4, GFP (GFP-injected 6-OHDA-treated mice) n=5, DX2 (DX2-injected 6-OHDA-treated mice) n=11 (each mice), scAAV; scAAV-GFP 4×10⁹ vg, scAAV-DX2 low 1.6×10⁸ vg, scAAV-DX2 4×10⁹ vg, ns; non-significant, *P<0.05, **P<0.005, ***P<0.001, t-test. FIG. 10D. Immunofluorescence image of GFP and DX2-injected mice brain. The white square box indicates TH positive dopaminergic neuronal cells and the white arrows shows indicated virus injection site. FIG. 10E. The survival rate in each mice group. Animals; n=15, Saline indicates saline-treated wild type mice. L-DOPA, GFP, and DX2 represent L-DOPA, GFP, and DX2 injection in 6-OHDA-treated mice. scAAV; scAAV-GFP (GFP) 4×10⁹ vg, scAAV-DX2 (DX2) (low) 1.6×10⁸ vg, scAAV-DX2 (DX2) (high) 4×10⁹ vg. FIGS. 10F and 10G. DX2 and Bax mRNA expression of naïve, 6-OHDA and DX2-treated mice. ***P<0.001, t-test. FIG. 10H. RNA in situ hybridization to identify the DX2 expressed cells in AAV-DX2 injected 6-OHDA mice model.

FIGS. 11A-11G. DX2 restores motor symptoms in MPTP-induced PD model. FIG. 11A. scAAV-DX2-treated mouse showed slightly longer latency to fall in the rotarod test when compared with that of vehicle (scAAV-GFP, GFP) indicating that scAAV-DX2 attenuated damage towards dopaminergic neurons. FIG. 11B. DX2-treated mouse showed improved locomotor activity based on the SHIRPA test. FIG. 11C. DX2-treated mice showed a relatively lower level of limb deficit. FIG. 11D. DX2-overexpressed mouse showed improved grooming rate when compared with vehicle control (GFP). FIG. 11E. Immunofluorescence image of TH-positive cells in the mouse substantia nigra. The white square box indicates the TH expressing regions. FIGS. 11F and 11G. The DX2 (FIG. 11F) and Bax (FIG. 11G) mRNA expression of the indicated mice brain. Naïve, GFP, and DX2 indicate saline-treated wild type mice, GFP-injected MPTP-treated mice, and DX2-injected MPTP-treated mice. Animals; Naive n=6, GFP n=9, DX2 n=12, scAAV; scAAV-GFP 4×10⁹ vg, scAAV-DX2 4×10⁹ vg, *P<0.05, **P<0.001, ***P<0.0001, t-test.

FIGS. 12A and 12B. Administration of DX2 delays the disease onset (FIG. 12A) and prolongs the lifespan (FIG. 12B) in Lou Gehrig's disease model. Animals; n=5.

FIG. 13 represents the cell morphology in bright field microscopy. Overexpression of DX2 in AAV-DX2-infected cells inhibits Aβ-O-mediated cell death. DX2 increases cell viability in Aβ-O-treated cells. SK-SY5Y cells were incubated with AAV-DX2 or AAV-GFP in the absence or presence of 25 μM of Aβ-O, after 48 hours, cells death was observed by microscopy. Original magnification images, ×40 (upper panel), ×100 (lower panel).

FIG. 14 shows quantification of cell death in FIG. 13. White box shows the percentage of cell death and black box indicates the percentage of cell viability.

FIG. 15 indicates the expression level of p53. DX2 expression plays an important role in neurotoxin-induced p53 expression. *AAV-DX2 (#1) and AAV-DX2 (#2) indicates produced AAV-DX2 virus in different batch.

FIGS. 16A-16D. Mutant SOD1 selectively interacts with KARS1. FIG. 16A. Binding affinity of Lex-KARS1 to B42-SOD1 WT and mutants G85R and G93A was tested by the yeast two-hybrid assay. FIG. 16B. HA-SOD1 WT, G85R, and G93A were transfected into HEK 293 cells and immunoprecipitation (IP) was performed with HA antibody. Levels of KARS1 and SOD1 were determined by immunoblotting. FIG. 16C. Binding affinity of KARS1 fragments to SOD1 mutants determined by the yeast two-hybrid assay. FIG. 16D. N2A cells were transfected with myc-KARS1 and SOD1 WT, G93A, G85RA. IP of myc-KARS1 was performed and immunoblotted for detection of AIMP2 and 67LR (laminin receptor).

FIGS. 17A-17F. Mutant SOD1 decreased 67 laminin receptor inducing anoikis. FIG. 17A. SK-N-SH cells were transfected with SOD1 WT and G93A. The cells were harvested and immunblotted for 67 laminin receptor (LR). FIG. 17B. Neural cells were transfected with SOD1 WT and G93A then seeded on 22×22 cover slip. The cells fixed and then treated with KARS1 or 67LR antibody and then, the images were taken by confocal microscopy. The white arrow indicates stained laminin receptor. FIG. 17C. To see the effect of SOD1 transfection of WT and G93A on migration, neural cells were loaded to the upper chamber and WT and G93A into the lower chamber of trans-well plate separated by the membrane with 8.0 μm pore size. The membrane excised and stained. FIG. 17D. Neural cells were transfected with SOD1 WT or G93A then treated with Laminin 1(LN1) for 0, 15, 30 and 60 min. The pERK and ERK levels were checked by western blot. FIG. 17E. Binding affinity of KARS1 to 67 LR in WT and mutant SOD1 expressed cells determined by the immunoprecipitation. FIG. 17F. SH-SY5Y cells were seeded transfected with KARS1 for 24 h and then SOD1 WT, G85R, and G93A for 24 h. Then they were seeded in a hema-coated then treated with TNF-α and cycloheximide (CHX) for 6 h. MTT assay was performed to observe cell viability.

FIGS. 18A-18D. The effects of AIMP2-DX2 gene on KARS1 and 67LR. FIG. 18A. SK-N-SH cells were transfected with SOD1 WT, or G93A then treated with KARS1 with DX2 or AIMP2. The cells were harvested and western blot was performed. FIG. 18B. Neuroblastoma cells were transfected with strep-DX2 for 24 h and then SOD1 WT, G93A and G85R for 24 h. The cells were harvested and subcellular fractionation was performed and the samples were immunoblotted. FIG. 18C. Neural cells were transfected with SOD1 WT, or G93A and then they were treated with AAV-EV or AAV-DX2. And the cells were treated with laminin 1 (LN1) for 0, 15, 30 and 60 min. The cells were lysed and then immunblotted for p-ERK and ERK levels. FIG. 18D. SH-SY5Y cells were transfected with SOD1 WT, or G93A then treated with TNF-α (30 ng/mL) for 24 h. The attachment of cells was measured by iCelligence.

FIGS. 19A-19B. Administration of DX2 rescue mutant SOD1 induced neuronal death. FIG. 19A. SH-SY5Y cells were transfected with SOD1 WT, G85R and G93A and treated with TNF-α and cycloheximide (CHX) for 6 h followed by adeno associated virus (AAV) control vector (GFP) or DX2. The cells viability was check by MTT assay. FIG. 19B. The primary neuronal cells were isolated in each mouse, seeded on 24-well plate, treated with AAV-GFP or AAV-DX2, and MTT assay was performed to check their viability.

FIG. 20A. A binding assay shows that DX2 binds to PARP-1 more strongly than AIMP2. FIG. 20B. AIMP2-transfected cells showed significantly increased cleavage of PARP-1 when compared to the expression seen in other transfected cells under oxidative stress conditions. However, PARP-1 cleavage was not observed in DX2-transfected cells. FIG. 20C. PARylation of AIMP2 was increased in the presence of H₂O₂, but the PARlylation of DX2 was not altered.

FIGS. 21A-21C. A comparison of the amino acid sequences of AIMP2-DX2 and variants (FIGS. 21B and 21C are continuations of FIG. 21A).

FIGS. 22A-22B. Inhibition of neuromuscular junction damage. In FIG. 22A, the neuromuscular junctions were stained with alpha-Bungarotoxin, and synaptic vesicle and end plate were staining with SV2 and 2H3. In FIG. 22B, the number of innervated endplates was counted and represented.

### DETAILED DESCRIPTION OF THE INVENTION

AIMP2-DX2 is an alternative, antagonistic splicing variant of AIMP2 (aminoacyl tRNA synthase complex-interacting multifunctional protein 2), which is a multifactorial apoptotic gene. AIMP2-DX2 is known to suppress cell apoptosis by hindering the functions of AIMP2.

AIMP2-DX2, acting as a competitive inhibitor of AIMP2, suppresses TNF-alpha mediated apoptosis through inhibition of ubiquitination/degradation of TRAF2. In addition, AIMP2-DX2 has been previously identified as a lung cancer-inducing factor.

It has also been determined that AIMP2-DX2 can treat neuronal diseases (US2019/0298858 A1).

Disclosed herein are methods for delaying disease onset of a subject with amyotrophic lateral sclerosis (ALS), comprising administering to the subject a recombinant vector comprising an exon 2-deleted AIMP2 variant (AIMP2-DX2) gene.

Disclosed herein are methods of inhibiting neuronal cell death in a subject with amyotrophic lateral sclerosis (ALS), comprising administering to the subject a recombinant vector comprising an exon 2-deleted AIMP2 variant (AIMP2-DX2) gene.

Disclosed herein are methods of treating muscle atrophy in a subject in need thereof, comprising administering to the subject a recombinant vector comprising an exon 2-deleted AIMP2 variant (AIMP2-DX2) gene. In some embodiments, the subject has amyotrophic lateral sclerosis (ALS). In some embodiments, the subject has spinal muscular atrophy (SMA).

Disclosed herein are methods for increasing survival rate or prolonging lifespan of a subject with Parkinson's disease (PD), comprising administering to the subject a recombinant vector comprising an exon 2-deleted AIMP2 variant (AIMP2-DX2) gene.

Disclosed herein are methods of preventing behavior deficit, restoring a motor symptom, and/or reducing neuronal damage in a subject with Parkinson's disease (PD), comprising administering to the subject a recombinant vector comprising an exon 2-deleted AIMP2 variant (AIMP2-DX2) gene.

Disclosed herein are methods of inhibiting amyloid beta oligomer (Aβ-O)-induced neuronal cell death or Aβ-O-induced p53 expression in a subject with Alzheimer's disease (AD), comprising administering to the subject a recombinant vector comprising an exon 2-deleted AIMP2 variant (AIMP2-DX2) gene.

Disclosed herein are methods of inhibiting neuromuscular junction (NMJ) damage in a subject with spinal muscular atrophy (SMA), comprising administering to the subject a recombinant vector comprising an exon 2-deleted AIMP2 variant (AIMP2-DX2) gene.

Disclosed herein are methods of inhibiting neuromuscular junction (NMJ) damage, inhibiting NMJ block induced respiratory failure, difficulty in breathing, inhibiting NMJ block induced muscle twitching or fasciculation, in a subject with amyotrophic lateral sclerosis (ALS), comprising administering to the subject a recombinant vector comprising an exon 2-deleted AIMP2 variant (AIMP2-DX2) gene.

Disclosed herein are methods of suppressing anoikis, and/or increasing laminin receptor stabilization in a subject with amyotrophic lateral sclerosis (ALS), Parkinson's disease (PD), comprising administering to the subject a recombinant vector comprising an exon 2-deleted AIMP2 variant (AIMP2-DX2) gene.

Also disclosed are methods of inhibiting inflammation in a subject with ALS, methods of preventing behavior deficit, inhibiting neuronal cell death, and/or muscle atrophy in a subject with PD, methods of restoring motor symptoms in a subject with PD, methods of treating Alzheimer's Disease (AD) in a subject with AD, and/or methods of treating congenital muscular dystrophy, Multiple sclerosis, Muscular dystrophy, Myasthenia gravis, Myopathy, Myositis (including polymyositis and dermatomyositis), Peripheral neuropathy, Spinal muscular atrophy, and/or other cell death induced CNS disease in a subject in need thereof, comprising administering to the subject a recombinant vector comprising an exon 2-deleted AIMP2 variant (AIMP2-DX2) gene.

The recombinant vector as disclosed herein can further comprise an miR-142 target sequence. The vector can further comprise a promoter operably linked to the AIMP2-DX2. In some embodiments, the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter, Synapsin promoter, MeCP2 promoter, CaMKII promoter, Hb9 promoter, or opsin promoter.

In the methods disclosed herein, in some embodiments, the recombinant vectors comprise an exon 2-deleted AIMP2 variant (AIMP2-DX2) gene and an miR-142 target sequence. The miR-142 target sequence can be 3′ to the AIMP2-DX2 gene. The vectors described herein can express AIMP2-DX2 in neuronal cells but not in hematopoietic cells, such as leukocytes and lymphoid cells. Thus, the vectors described herein can be useful in specifically targeting neuronal cells for treating neuronal diseases.

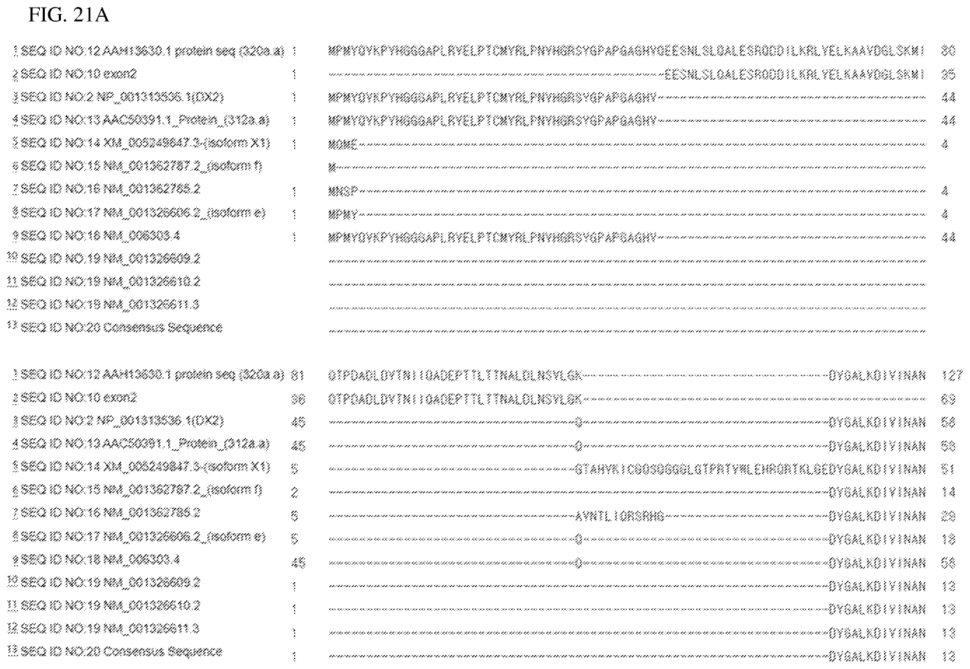

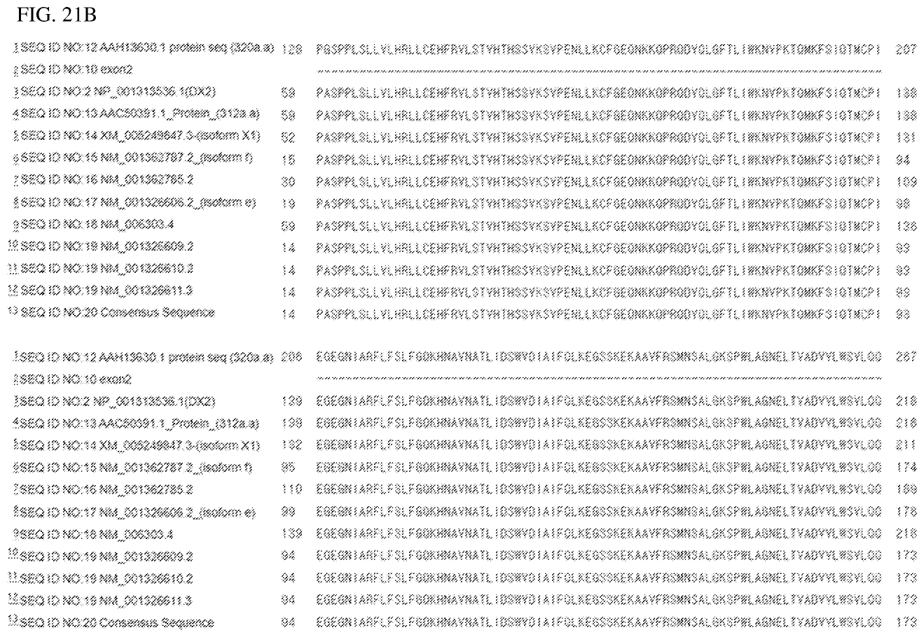

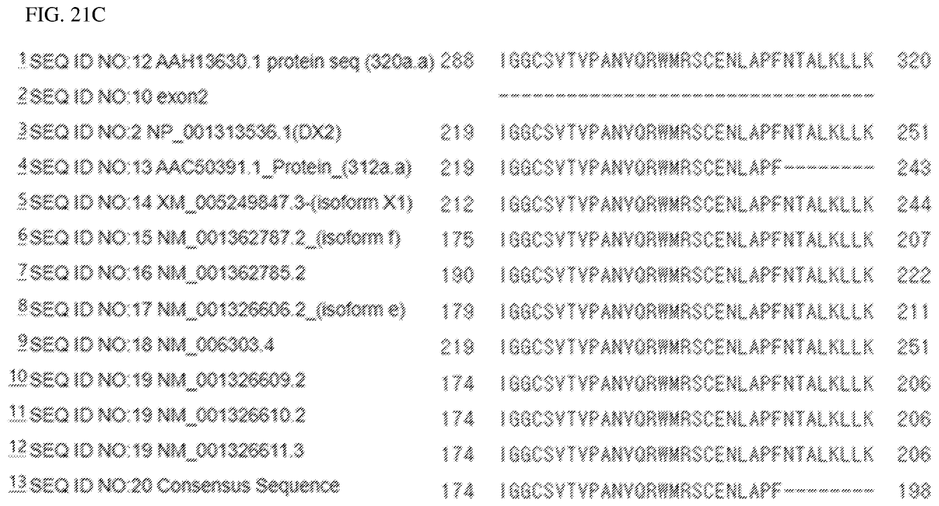

The AIMP2-DX2 polypeptide (SEQ ID NO:2) is a splice variant of AIMP2 (e.g., aa sequence of SEQ ID NO:12; e.g., nt sequence of SEQ ID NO:3), in which the second exon (SEQ ID NO:10; nt sequence of SEQ ID NO:4) of AIMP2 is omitted. In some embodiments, the AIMP2-DX2 gene has a nucleotide sequence set forth in SEQ ID NO:1, and the AIMP2-DX2 polypeptide has an amino acid sequence set forth in SEQ ID NO:2. Variants or isoforms of the AIMP2-DX2 polypeptide are also known and can be determined by those in the art (see, e.g., SEQ ID NOS:13-19). For example, FIGS. 21A-21C show a comparison of AIMP2 (SEQ ID NO:2) and variants, SEQ ID NO:13-19, as well as a consensus or core sequence (SEQ ID NO:20).

In some embodiments, the AIMP2-DX2 gene can comprise a nucleotide sequence encoding an amino acid sequence that is at least 90% identical, at least 93% identical, at least 95% identical, at least 96% identical, at least 97% identical, at least 98% identical, at least 99% identical to SEQ ID NO:2, 13, 14, 15, 16, 17, 18, 19, or 20, or any ranges of % identity therein. The AIMP2-DX2 gene can comprise a nucleotide sequence encoding an amino acid sequence of SEQ ID NO:2, 13, 14, 15, 16, 17, 18, 19, or 20.

The AIMP2-DX2 gene can comprise a nucleotide sequence at least 90% identical, at least 93% identical, at least 95% identical, at least 96% identical, at least 97% identical, at least 98% identical, at least 99% identical to a nucleotide sequence of SEQ ID NO:1, or any ranges of % identity therein. The AIMP2-DX2 gene can comprise a nucleotide sequence of SEQ ID NO:1.

In some embodiments, the AIMP2-DX2 gene does not have an exon comprising a nucleotide sequence encoding an amino acid sequence having at least 70%, at least 75%, at least 80%, at least 85%, at least 90%, at least 95%, at least 96%, at least 97%, at least 98%, at least 99%, or 100% identity to SEQ ID NO:10 or 11. In some embodiments, the AIMP2-DX2 gene does not have an exon comprising a nucleotide sequence encoding an amino acid sequence of SEQ ID NO:10 or 11. In some embodiments, the AIMP2-DX2 gene does not have an exon comprising a nucleotide sequence having at least 70%, at least 75%, at least 80%, at least 85%, at least 90%, at least 95%, at least 96%, at least 97%, at least 98%, at least 99%, or 100% identity to SEQ ID NO:4.

The miR-142 target sequence (miR-142T) can comprise a nucleotide sequence comprising ACACTA. The miR-142 target sequence can comprise a nucleotide sequence comprising ACACTA and 1-17 additional contiguous nucleotides of SEQ ID NO:5. For example, the miR-142 target sequence can comprise a nucleotide sequence comprising ACACTA and a sum of 1, 2, 3, 4, 5, 6, 7, 8, 9, 10, 11, 12, 13, 14, 15, 16, or 17 additional nucleotides that are contiguous 5′ or 3′ of ACACTA as shown in SEQ ID NO:5.

The miR-142 target sequence can comprise a nucleotide sequence at least 50% identical, at least 60% identical, at least 70% identical, at least 80% identical, at least 90% identical, at least 90% identical, at least 93% identical, at least 95% identical, at least 96% identical, at least 97% identical, at least 98% identical, at least 99% identical, or 100% identical to a nucleotide sequence of SEQ ID NO:5 (TCCATAAAGTAGGAAACACTACA; miR-142-3pT). The miR-142 target sequence can comprise a nucleotide sequence of SEQ ID NO:5.

The miR-142 target sequence can comprise a nucleotide sequence comprising ACTTTA. The miR-142 target sequence can comprise a nucleotide sequence comprising ACTTTA and 1-15 additional contiguous nucleotides of SEQ ID NO:7. For example, the miR-142 target sequence can comprise a nucleotide sequence comprising ACTTTA and a sum of 1, 2, 3, 4, 5, 6, 7, 8, 9, 10, 11, 12, 13, 14, or 15 additional nucleotides that are contiguous 5′ or 3′ of ACTTTA as shown in SEQ ID NO:7.

The miR-142 target sequence can comprise a nucleotide sequence at least 50% identical, at least 60% identical, at least 70% identical, at least 80% identical, at least 90% identical, at least 90% identical, at least 93% identical, at least 95% identical, at least 96% identical, at least 97% identical, at least 98% identical, at least 99% identical, or 100% identical to a nucleotide sequence of SEQ ID NO:7 (AGTAGTGCTTTCTACTTTATG; miR-142-5pT). The miR-142 target sequence can comprise a nucleotide sequence of SEQ ID NO:7.

An example miR142-3pT mutant sequence is:

(SEQ ID NO: 25)
Ccgctgcagtgtgacagtgccagccaatgtgcaga
ggtggatgaggtcttgtgaaaacctggctcctttt
aacacggccctcaagctccttaagtgaccagaagc
ttgctagctccataaagtaggaCCACTGCAatcac
tccataaagtaggaCCACTGCAagatatctccata
aagtaggaCCACTGCAatcactccataaagtagga
CCACTGCAaaagcttgtagggatccgcc.

A mutant sequence refers to one or more regions, e.g., four regions, of core sequences of miR142 3pT that are substituted as follows: (5′-AACACTAC-3′→5′-CCACTGCA-3′). Inhibition of DX2 expression in vector transfected HEK293 cells was observed with the miR142-3p ×1 repeat (100 pmol) miR142-3p target sequence and as the number of core binding sequence in miR142-3p target seq are increased, miR142-3p inhibition on DX2 expression was also increased. The Tseq ×3 core sequence containing vector showed significant inhibition, whereas no inhibition was observed for the mutated 3× sequence.

A microRNA (miRNA) is a non-coding RNA molecule that functions to control gene expression. MiRNAs function via base-pairing with complementary sequences within mRNA molecules, i.e., a miRNA target sequences. miRNAs can bind to target messenger RNA (mRNA) transcripts of protein-coding genes and negatively control their translation or cause mRNA degradation. At present, more than 2000 human miRNAs have been identified and miRbase databases are publicly available. Many miRNAs are expressed in a tissue-specific manner and have an important roles in maintaining tissue-specific functions and differentiation.

MiRNA acts at the post-transcription stage of the gene and, in the case of mammals, and it is known that approximately 60% of the gene expression is controlled by miRNA. miRNA plays an important role in a diverse range of processes within living body and has been disclosed to have correlation with cancer, cardiac disorders and nerve related disorders. For example, miR-142-3p and miR-142-5p exist in miR-142 and any of the target sequences thereof can be used. Thus, “miR-142” or “miRNA-142” refers to, e.g., miR-142-3p and/or miR-142-5p, and can bind to the miR-142 target sequence, e.g., miR-142-3pT or miR-142-5pT.

The miR-142 target sequence can be 5′ or 3′ to the AIMP2-DX2 gene.

For example, “miR-142-3p” can exist in the area at which translocation of its gene occurs in aggressive B cell leukemia and is known to express in hemopoietic tissues (bone marrow, spleen and thymus, etc.). In addition, miR-142-3p is known to be involved in the differentiation of hemopoietic system with confirmation of expression in the liver of fetal mouse (hemopoietic tissue of mouse).

In some embodiments, the miR-142-3p and/or miR-142-5p target sequence is repeated at least 2-10 times, at least 2-8 times, at least 2-6 times, at least 4 times, or any range or number of times thereof.

As an example, the miR-142-3p, e.g., having a nucleotide sequence of SEQ ID NO:23, can have a corresponding target sequence, e.g., a miR-142-3p target sequence (miR-142-3pT) having a nucleotide sequence of SEQ ID NO:5 but not limited thereto. The miR-142-5p, e.g., having a nucleotide sequence of SEQ ID NO:24 can have a corresponding target sequence, e.g., a miR-142-5p target sequence (miR-142-5pT) having a nucleotide sequence of SEQ ID NO:7 but not limited thereto.

In some embodiments, an miR-142-3p can have a nucleotide sequence of SEQ ID NO:23 and an miR-142-5p can have a nucleotide sequence of SEQ ID NO:24.

Disclosed herein are recombinant vectors that can control the side effect of over-expression of the AIMP2-DX2 variant in a tumor by inserting an miR-142-3p and/or miR-142-5p target sequence (miR-142-3pT and/or miR-142-5pT, respectively) into a terminal end of AIMP2-DX2 and controlling suppression of AIMP2-DX2 expression in CD45-derived cells, in particular, the lymphatic system and leukocytes. Thus, the expression of AIMP2-DX2 variant can be restricted to only in the injected neuronal cells and tissues but not in non-neuronal hematopoietic cells, the major population in the injected tissue areas. MiR142-3p is expressed only in hematopoietic cells.

Disclosed herein are recombinant vectors containing a target sequence for miR-142-3p and/or miR-142-5p. Disclosed herein are recombinant vectors comprising an exon 2-deleted AIMP2 variant (AIMP2-DX2) gene and miR-142-3p and/or miR-142-5p target sequences as disclosed herein.

The term “recombinant vector” refers to vector that can be expressed as the target protein or RNA in appropriate host cells, and gene construct that contains essential operably linked control factor to enable the inserted gene to be expressed appropriately.

The term “operably linked” refers to functional linkage between the nucleic acid expression control sequence and nucleic acid sequence that codes the targeted protein and RNA to execute general functions. For example, it can affect the expression of nucleic acid sequence that codes promoter and protein or RNA that has been linked for operability of the nucleic acid sequence. Operable linkage with recombinant vector can be manufactured by using gene recombinant technology, which is known well in the corresponding technology area, and uses generally known enzymes in the corresponding technology area for the area-specific DNA cutting and linkage.

The recombinant vectors can further comprise a promoter operably linked to a AIMP2-DX2 as disclosed herein. In some embodiments, the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter, Synapsin promoter, MeCP2 promoter, CaMKII promoter, Hb9 promoter, or opsin promoter.

The recombinant vector can additionally contain heterogeneous promoter and operably linked heterogeneous gene in the promoter.

“Heterogeneous gene” as used herein can include protein or polypeptide with biologically appropriate activation, and encrypted sequence of the targeted product such as immunogen or antigenic protein or polypeptide, or treatment activation protein or polypeptide.

Polypeptides can supplement deficiency or absent expression of endogenous protein in host cells. The gene sequence can be induced from a diverse range of suppliers including DNA, cDNA, synthesized DNA, RNA or its combinations. The gene sequence can include genome DNA that contains or does not contain natural intron. In addition, the genome DNA can be acquired along with promoter sequence or polyadenylated sequence. Genome DNA or cDNA can be acquired in various methods. genome DNA can be extracted and purified from appropriate cells through method publicly notified in the corresponding area. Or, mRNA can be used to produce cDNA by reverse transcription or other method by being separated from the cells. Or, polynucleotide sequence can contain sequence that is complementary to RNA sequence, for example, antisense RNA sequence, and the antisense RNA can be administered to individual to suppress expression of complementary polynucleotide in the cells of individuals.

For example, the heterogeneous gene is an AIMP-2 splicing variant with the loss of exon 2 and miR-142-3p target sequence can be linked to 3′ UTR of the heterogeneous gene. The sequence of the AIMP2 protein (312aa version: AAC50391.1 or GI: 1215669; 320aa version: AAH13630.1, GI: 15489023, BC0 13630.1) are described in the literatures (312aa version: Nicolaides, N. C., Kinzler, K. W. and Vogelstein, B. Analysis of the 5′ region of PMS2 reveals heterogeneous transcripts and a novel overlapping gene, Genomics 29 (2), 329-334 (1995)/320 aa version: Generation and initial analysis of more than 15, 000 full-length human and mouse cDNA sequences, Proc. Natl. Acad. Sci. U.S.A. 99 (26), 16899-16903 (2002)).

The term “AIMP2 splicing variant” refers to the variant generated due to partial or total loss of exon 2 among exons 1 to 4. As such, the variant signifies interference of the normal function of AIMP2 by forming AIMP2 protein and heterodimer. The injected AIMP2-DX2 gene is rarely expressed in tissues other than the injected tissue. However, as an additional safety measure, an miR142 target sequence can be inserted to completely block the possibility of AIMP2-DX2 being expressed in hematopoietic cells, the major population of non-neuronal cells in the injected tissue area.

The recombinant vector can include SEQ ID NOS:1 and 5.

The term “% of sequence homology,” “% identity,” or “% identical” to a nucleotide or amino acid sequence can be, e.g., confirmed by comparing the 2 optimally arranged sequence with the comparison domain and some of the nucleotide sequences in the comparison domain can include addition or deletion (that is, gap) in comparison to the reference sequence on the optimal arrange of the 2 sequences (does not include addition or deletion).

Protein as disclosed herein not only includes those with its natural type amino acid sequence but also those with variant amino acid sequences.

Variants of the protein signify proteins with difference sequences due to the deletion, insertion, non-conservative or conservative substitution or their combinations of the natural amino acid sequence and more than 1 amino acid residue. Amino acid exchange in protein and peptide that does not modify the activation of the molecule in overall is notified in the corresponding area (H. Neurath, R. L. Hill, The Proteins, Academic Press, New York, 1979).

The protein or its variant can be manufactured through natural extraction, synthesis (Merrifield, J. Amer. Chem. Soc. 85: 2149-2156, 1963) or genetic recombination on the basis of the DNA sequence (Sambrook et al, Molecular Cloning, Cold Spring Harbour Laboratory Press, New York, USA, 2^{nd} Ed., 1989).

Amino acid mutations can occur on the basis of the relative similarity of the amino acid side chain substituent such as hydrophilicity, hydrophobicity, electric charge and size, etc. In accordance with the analysis of the size, shape and types of amino acid side chain substituent, it can be discerned that arginine, lysine and histidine are residues with positive charge; alanine, glycine and serine have similar sizes; phenylalanine, tryptophan and tyrosine have similar shapes. Therefore, on the basis of such considerations, arginine, lysine and histidine; alanine, glycine and serine; and phenylalanine, tryptophan and tyrosine can be deemed functional equivalents biologically.

In introducing one or more mutations, hydrophobic index of amino acid can be considered. Hydrophobic index is assigned to each amino acid according to hydrophobicity and charge: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5)

In assigning interactive biological function of protein, hydrophobic amino acid index is very important. It is possible to have similar biological activation only if a substitution is made with an amino acid with a similar hydrophobic index. In the event of introducing a mutation by making reference to the hydrophobic index, substitution between amino acids with hydrophobic index differences within ±2, within ±1, or within ±0.5.

Meanwhile, it is also well known that substitution between amino acids with similar hydrophilicity value can induce proteins with equivalent biological activation. As indicated in U.S. Pat. No. 4,554,101, the following hydrophilic values are assigned to each of the amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine 31 1.8); isoleucine (−1.8); tyrosine (−2.3); phenylalanine (−2.5); tryptophan (−3.4).

In the event of introducing one or more mutations by making reference to hydrophilic values, substitutions can be made between amino acids with hydrophilic value differences within ±2, within ±1, or within ±0.5. but not limited thereto.

Amino acid exchange in protein that does not modify the activation of molecule in overall is notified in the corresponding area (H. Neurath, R. L. Hill, The Proteins, Academic Press, New York, 1979). The most generally occurring exchanges are those between the amino acid residues including Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu and Asp/Gly. Vector system can be constructed through diverse methods announced in the corresponding industry. The specific methods are described in Sambrook et al. (2001), Molecular Cloning, A Laboratory Manual, Cold Spring Harbor Laboratory Press.

Vectors disclosed herein can be constructed as a typical vector for cloning or for expression. In addition, the vectors can be constructed with prokaryotic or eukaryotic cells as the host. If the vector is an expression vector and prokaryotic cell is used as the host, it is general to include powerful promoter for execution of transcription (for example, tac promoter, lac promoter, lacUV5 promoter, 1pp promoter, pL X promoter, pRX promoter, rac5 promoter, amp promoter, recA promoter, SP6 promoter, trp promoter and T7 promoter, etc.), ribosome binding site for commencement of decoding and transcription/decoding termination sequence. In the case of using E. coli (for example, HB101, BL21, DH5a, etc.) as the host cell, promoter and operator site of the tryptophan biosynthesis route of E. coli (Yanofsky, C.(1984), J. Bacteriol., 158: 1018-1024) and left directional promoter of phage X (pLX promoter, Herskowitz, I. and Hagen, D. (1980), Ann. Rev. Genet., 14: 399-445) can be used as the control site.

Meanwhile, vectors that can be used can be more than 1 type, such as a virus vector, linear DNA, or plasmid DNA.

“Virus vector” refers to a virus vector capable of delivering gene or genetic substance to the desired cells, tissue and/or organ.

Although the virus vectors can include more than 1 species from the group composed of Adenovirus, Adeno-associated virus, Lentivirus, Retrovirus, HIV (Human immunodeficiency virus), MLV (Murine leukemia virus), ASLV (Avian sarcoma/leukosis), SNV (Spleen necrosis virus), RSV (Rous sarcoma virus), MMTV (Mouse mammary tumor virus) and Herpes simplex virus, it is not limited thereto. In some embodiments, the viral vector can be an adeno-associated virus (AAV), adeonovirus, lentivirus, retrovirus, vaccinia virus, or herpes simplex virus vector.

Although Retrovirus has an integration function for the genome of host cells and is harmless to the human body, it can have characteristic including suppressing the functions of normal cells at the time of integration, ability to infect a diverse range of cells, ease of proliferation, accommodate approximately 1-7 kb of external gene and generate duplication deficient virus. However, Retroviruses can also have disadvantages including difficulties in infecting cells after mitotic division, gene delivery under an in vivo condition and need to proliferate somatic cells under in vitro condition. In addition, Retroviruses have the risk of spontaneous mutations as it can be integrated into proto-oncogene, thereby presenting the possibility of cell necrosis.

Meanwhile, Adenoviruses have various advantages as a cloning vector including duplication even in nucleus of cells in medium level size, clinically nontoxic, stable even if external gene is inserted, no rearrangement or loss of genes, transformation of eukaryotic organism and stably undergoes expression at high level even when integrated into host cell chromosome. Good host cells of Adenoviruses are the cells that are the causes of hemopoietic, lymphatic and myeloma in human. However, proliferation is difficult since it is a linear DNA and it is not easy to recover the infected virus along with low infection rate of virus. In addition, expression of the delivered gene is most extensive during 1-2 weeks with expression sustained over the 3-4 weeks only in some of the cells. Another issue is that it has high immuno-antigenicity.

Adeno-associated virus (AAV) has been preferred in recent years since it can supplement the aforementioned problems and has a lot of advantages as gene therapy agent. It is also referred as adenosatellite virus. Diameter of adeno-associated virus particle is 20 nm and is known to have almost no harm to human body. As such, its sales as gene therapy agent in Europe were approved.

AAV is a provirus with single strand that needs auxiliary virus for duplication and AAV genome has 4,680 bp that can be inserted into specific area of the chromosome 19 of the infected cells. Trans-gene is inserted into the plasma DNA connected by the 2 inverted terminal repeat (ITR) sequence section with 145 bp each and signal sequence section. Transfection is executed along with other plasmid DNA that expresses the AAV rep and cap sections, and Adenovirus is added as an auxiliary virus. AAV has the advantages of wide range of host cells that deliver genes, little immunological side effects at the time of repetitive administration and long gene expression period. Moreover, it is safe even if the AAV genome is integrated with the chromosome of host cells and does not modify or rearrange the gene expression of the host.

The Adeno-associated virus is known to have a total of 4 serotypes. Among the serotypes of many Adeno-associated viruses that can be used in the delivery of the target gene, the most widely researched vector is the Adeno-associated virus serotype 2 and is currently used in the delivery of clinical genes of cystic fibrosis, hemophilia and Canavan's disease. In addition, recently, the potential of recombinant adeno-associated virus (rAAV) is increasing in the area of cancer gene therapy. It was also the Adeno-associated virus serotype 2 that was used in the invention. Although it is possible to select and apply appropriate viral vector, it is not limited to this.

In addition, if the vectors are expression vectors and use eukaryotic cells as the host, promoter derived from the genome of mammalian cells (example: metallothionein promoter) or promoter derived from mammalian virus (example: post-adenovirus promoter, vaccine virus 7.5K promoter, SV40 promoter, cytomegalovirus promoter and HSV TK promoter) can be used. Specifically, although it can include more than 1 species selected from the group composed of promoters selected from the group composed of LTR of Retrovirus, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter and opsin promoter, it is not limited to these. Moreover, it generally has polyadenylated sequence as the transcription termination sequence.

Vectors disclosed herein can be fused with other sequences as need to make the purification of the protein easier. Although the fused sequence such as glutathione S-transferase (Pharmacia, USA), maltose binding protein (NEB, USA), FLAG (MI, USA) and 6xHis (hexahistidine; Quiagen, USA), etc. can be used, for example, it is not limited to these. In addition, expression vectors can include tolerance gene against antibiotics generally used in the corresponding industry as the selective marker including Ampicillin, Gentamycin, Carbenicillin, Chloramphenicol, Streptomycin, Kanamycin, Geneticin, Neomycin and Tetracycline, as examples.

In addition, disclosed herein are gene carriers including the recombinant vector containing a target sequence (miR-142-3pT and/or miR-142-5pT) for miR-142, such as miR-142-3p and/or miR-142-5p, respectively.

The term “gene transfer” includes delivery of genetic substances to cells for transcription and expression in general. Its method is ideal for protein expression and treatment purposes. A diverse range of delivery methods such as DNA transfection and virus transduction are announced. It signifies virus-mediated gene transfer due to the possibility of targeting specific receptor and/or cell types through high delivery efficiency and high level of expression of delivered genes, and, if necessary, nature-friendliness or pseudo-typing.

The gene carriers can be transformed entity that has been transformed into the recombinant vector, and transformation includes all methods of introducing nucleic acid to organic entity, cells, tissues or organs, and as announced in the corresponding area, it is possible to select and execute appropriate standard technology in accordance with the host cells. Although such methods include electroporation, fusion of protoplasm, calcium phosphate (CaP0₄) sedimentation, calcium chloride (CaCl₂) sedimentation, mixing with the use of silicone carbide fiber, agribacteria-mediated transformation, PEG, dextran sulphate and lipofectamin, etc., it is not limited to these.

The gene carriers are for the purpose of expression of heterogeneous genes in neuron. As such it suppresses the expression of the heterogeneous gene in CD45-derived cells and can increase the expression of heterogeneous gene in brain tissue. Majority of the CD45 are transmembrane protein tyrosine phosphatase situated at the hematopoietic cell. Cells can be defined in accordance with the molecules situated on the cell surface and the CD45 is the cell marker for all leukocyte groups and B lymphocytes. The gene carrier is not be expressed in the CD45-derived cells, in particular, in lymphoid and leukocyte range of cells.

The gene carriers can additionally include carrier, excipient or diluent allowed to be used pharmacologically.

In addition, disclosed herein are methods of delivering and expressing the heterogeneous gene in the neuron that includes the stage of introducing the recombinant vector into the corresponding entity.

The methods can increase the expression of heterogeneous gene in cerebral tissues and control heterogeneous gene expression in other tissues.

In addition, disclosed herein are vectors comprising 1) a promoter; 2) a nucleotide sequence that encodes a target protein linked with the promoter to enable operation; and 3) an expression cassette that includes the nucleotide sequence targeting miR-142-3p inserted into 3′UTR of the nucleotide sequence. In some embodiments, the vectors can comprise 1) a promoter; 2) a nucleotide sequence that encodes a target protein linked with the promoter to enable operation; and 3) an expression cassette that includes the nucleotide sequence targeting miR-142-5p inserted into 3′UTR of the nucleotide sequence.

The term “expression cassette” refers to the unit cassette that can execute expression for the production and secretion of the target protein operably linked with the downstream of signal peptide as it includes a gene that encodes the target protein and a nucleotide sequence that encodes the promoter and signal peptide. Secretion expression cassette of the invention can be used mixed with the secretion system. A diverse range of factors that can assist the efficient production of the target protein can be included in and out of such expression cassette.

In addition, disclosed herein are preventive or therapeutic preparations for neurodegenerative diseases that include a nucleotide sequence that encodes AIMP-2 splicing variant with loss of exon 2 and a nucleotide sequence that targets miR-142-3p linked to 3′UTR of the nucleotide sequence.

Accordingly, also disclosed herein are methods of treating a neuronal disease in a subject in need thereof, comprising administering any of the vectors disclosed herein. Although the neurodegenerative diseases can be more than 1 of the diseases selected from the group composed of Alzheimer's disease, Parkinson's disease, amyotrophic lateral sclerosis (ALS), retinal degeneration, mild cognitive impairment, multi-infarct dementia, fronto-temporal dementia, dementia with Lewy bodies, Huntington's disease, degenerative neural disease, metabolic cerebral disorders, depression, epilepsy, multiple sclerosis, cortico-basal degeneration, multiple system atrophy, progressive supranuclear palsy, dentatorubropallidoluysian atrophy, spinocerebella ataxia, primary lateral sclerosis, spinal muscular atrophy and stroke, it is not limited to these. In some embodiments, the neuronal disease is ALS. The treatment can improve memory, dyskinesia, motor activity, and/or prolong lifespan of the subject with a neuronal disease, e.g., ALS, Alzheimer's disease, or Parkinson's disease. In some embodiments, the treatment can improve motor activity and/or prolong lifespan of the subject with a neuronal disease, e.g., ALS.

The vectors disclosed herein can effect, but not limited to, apoptosis inhibition, dyskinesia amelioration, and/or oxidative stress inhibition, and thus prevent or treat neuronal diseases.

The term “treatment” includes not only complete treatment of neurodegenerative diseases but also partial treatment, improvement and/or reduction in the overall symptoms of neurodegenerative diseases as results of application of the pharmacological agents disclosed herein.

The term “prevention” signifies prevention of the occurrence of overall symptoms of neurodegenerative diseases in advance by suppressing or blocking the symptoms or phenomenon such as cognition disorder, behavior disorder and destruction of brain nerves by applying pharmacological agents disclosed herein.

Adjuvants other than the active ingredients can be included additionally to the pharmacological agents disclosed herein. Although any adjuvant can be used without restrictions as long as it is known in the corresponding technical area, it is possible to increase immunity by further including complete and incomplete adjuvant of Freund, for example.

Pharmacological agents disclosed herein can be manufactured in the format of having mixed the active ingredients with the pharmacologically allowed carrier. Here, pharmacologically allowed carrier includes carrier, excipient and diluent generally used in the area of pharmacology. Pharmacologically allowed carrier that can be used for the pharmacological agents disclosed herein include lactose, dextrose, sucrose, sorbitol, mannitol, xylitol, erythritol, malitol, starch, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil, but not limited to these.

Pharmacological agents disclosed herein can be used by being manufactured in various formats including oral administration types such as powder, granule, pill, capsule, suspended solution, emulsion, syrup and aerosol, etc., and external application, suppository drug or disinfection injection solution, etc. in accordance with their respective general manufacturing methods.

When manufactured into preparations, diluents or excipients such as filler, extender, binding agent, humectant, disintegrating agent and surfactant, etc., which are used generally, can be used in the manufacturing. Solid preparations for oral administration include pill, tablet, powder, granule and capsule preparations, and such solid preparations can be manufactured by mixing more than 1 excipient such as starch, calcium carbonate, sucrose, lactose and gelatin with the active ingredients. In addition, lubricants such as magnesium stearate and talc can also be used in addition to simple excipients. Liquid preparations for oral administration include suspended solution, solution for internal use, oil and syrup, etc. with the inclusion of various excipients such as humectant, sweetening agent, flavoring and preservative, etc. other than water and liquid paraffin, which are the generally used diluents. Preparations for non-oral administration include sterilized aqueous solution, non-aqueous solvent, suspension agent, oil, freeze dried agent and suppository. Vegetable oil such as propylene glycol, polyethylene glycol and olive oil, and injectable esters such as ethylate can be used as non-aqueous solvent and suspension solution. Agents for suppository can include witepsol, tween 61, cacao oil, laurine oil and glycerogelatin, etc.

Pharmacological agents can be administered into a subject or entity through diversified channels. All formats of administration such as oral administration, and intravenous, muscle, subcutaneous and intraperitoneal injection can be used.

Desirable doses of administration of therapeutic agents disclosed herein differ depending on various factors including preparation production method, administration format, age, weight and gender of the patient, extent of the symptoms of the disease, food, administration period, administration route, discharge speed and reaction sensitivity, etc. Nonetheless, it can be selected appropriately by the corresponding manufacturer. However, for the treatment effects, skilled medical doctor can determine and prescribe effective dose for the targeted treatment. For example, the treatment agents include intravenous, subcutaneous and muscle injection, and direction injection into cerebral ventricle or spinal cord by using micro-needle. Multiple injections and repetitive administrations are possible, e.g., the effective dose is 0.05 to 15 mg/kg in the case of vector, 5×10¹¹ to 3.3×10¹⁴ viral particle (2.5×10¹² to 1.5×10¹⁶ IU)/kg in the case of recombinant virus and 5×10² to 5×10⁷cells/kg in the cells. Desirably, the doses are 0.1 to 10 mg/kg in the case of vector, 5×10¹² to 3.3×10¹³ particles (2.5×10¹³ to 1.5×10¹⁵ IU)/kg in the case of recombinant virus and 5×10³ to 5×10⁶ cells/kg in the case of cells at the rate of 2 to 3 administrations per week. The dose is not strictly restricted. Rather, it can be modified in accordance with the condition of the patient and the extent of manifestation of the neural disorders. Effective dose for other subcutaneous fat and muscle injection, and direct administration into the affected area is 9×10¹⁰ to 3.3×10¹⁴ recombinant viral particles with the interval of 10 cm and at the rate of 2-3 times per week. The dose is not strictly restricted. Rather, it can be modified in accordance with the condition of the patient and the extent of manifestation of the neural disorders. More specifically, pharmacological agent in accordance with the invention includes 1×10¹⁰ to 1×10¹² vg(virus genome)/mL of recombinant adeno-associated virus and, generally, it is advisable to inject 1×10¹² vg once every 2 days over 2 weeks. It can be administered once a day or by dividing the dose for several administrations throughout the day. In some embodiments, the vectors can be administered in a dose of 0.1×10⁸ vg to 500×10⁸ vg, 1×10⁸ vg to 100×10⁸ vg, 1×10⁸ vg to 10×10⁸ vg, e.g., 5×10⁸ vg, or any ranges derived therefrom. For IV injections, e.g., vg can be translated to doses for human based on body weight for IV injection. For local tissue injections, e.g., vg can also be translated to doses for humans based on the target cell number and effective MOI (multiplicity of infection).

In some embodiments, the vectors disclosed herein can be injected to a subject by, e.g., subretinal injection, intravitreal injection, or subchoroidal injection. The injection can be in the form of a liquid. In other embodiments, the vectors disclosed herein can be administered to a subject in the form of eye drops or ointment.

The pharmacological preparations can be produced in a diverse range of orally and non-orally administrable formats. In some embodiments, the vector disclosed herein can be administered to the brain or spinal cord. In some embodiments, the vectors disclosed herein can be administered to the brain by stereotaxic injection.

Orally administrative agents include pills, tablets, hard and soft capsules, liquid, suspended solution, oils, syrup and granules, etc. These agents can include diluent (example: lactose, dextrose, sucrose, mannitol, sorbitol, cellulose and/or glycine) and glydents (example: silica, talc, and stearic acid and its magnesium or calcium salts, and/or polyethylene glycol) in addition to the active ingredients. In addition, the pills can contain binding agents such as magnesium aluminum silicate, starch paste, gelatin, tragacanthin, methyl cellulose, sodium carboxymethyl cellulose and/or polyvinyl pyrrolidine, and, depending on the situation, can contain disintegration agent such as starch, agar, alginic acid or its sodium salt or similar mixture and/or absorbent, coloring, flavor and sweetener. The agents can be manufactured by general mixing, granulation or coating methods.

In addition, injection agents are the representative form of non-orally administered preparations. Solvents for such injection agents include water, Ringer's solution, isotonic physiological saline and suspension. Sterilized fixation oil of the injection agent can be used as solvent or suspension medium, and any non-irritating fixation oil including mono- and di-glyceride can be used for such purpose. In addition, the injection agent can use fatty acids such as oleic acid.

The invention will be explained in more detail by using the following execution examples below. However, the following execution examples are only for the purpose of specifying the contents of the invention and do not limit the application of the invention to such examples.

### EXAMPLES

### Example 1. Production of the Recombinant Vector

Majority of CD45 are transmembrane protein tyrosine phosphatase of the hematopoietic cell, which can be used to define the cells in accordance with the molecule on the cell surface. CD45 is a marker for all leukocyte groups and B lymphocytes. A recombinant vector has been produced that is expressed specifically and only in neurons without being expressed in CD45-derived cells, in particular, lymphoid and leukocyte cells. The recombinant vector contains a splicing variant in which exon 2 of the Aminoacyl tRNA Synthetase Complex Interacting Multifunctional Protein 2 (AIMP2) has been deleted and an miRNA capable of controlling the expression of the AIMP2 splicing variant.

As a distribution safety measure, the recombinant vector was produced as above in order to induce specific expression of the AIMP2 splicing variant only in injected neuronal tissues and to completely block the possibility of AIMP2-DX2 being expressed in hematopoietic cells, the major population of non-neuronal cells in the injected tissue area.

### Example 1.1. Production of AIMP2 Variant

AIMP2 is one of the proteins involved in the formation of aminoacyl-tRNA synthetase (ARSs) and acts as a multifactorial apoptotic protein. In order to construct a plasmid that expresses the variant in which exon 2 of the AIMP2 has been deleted, cDNA of AIMP2 splicing variant was cloned into pcDNA3.1-myc. The sub-cloning in pcDNA3.1-myc was executed by using EcoRl and Xho1 after having amplified the AIMP2 splicing variant by using a primer having EcoR1 and Xho1 linker attached to the H322 cDNA.

AIMP2 variant having a nucleotide sequence of SEQ ID NO:1 and an amino acid sequence of SEQ ID NO:2 was used.

### Example 1.2. Sorting of miRNA and Selection of Its Target Sequence

As mentioned above, as a distribution safety measure, the recombinant vector was produced as above in order to confine the expression of the AIMP2 variant in injected neuronal cells and to completely block the possibility of AIMP2-DX2 being expressed in hematopoietic cells, the major population of non-neuronal cells in the injected tissue area.

For this purpose, miR-142-3p that is specifically expressed only in hematopoietic cells that generate leukocyte and lymphoid related cells was selected as the target. In order to produce a sequence that targets only the miR-142-3p, microarray data of mouse B cells and computer programming of genes targeted by miR⁻-142-3p (mirSVR score) were used. The miR-142-3p is a base sequence indicated with the sequence number of 3. The sequence targeting miR-142-3p was indicated with base sequence number of 4 that binds with miR-142-3p complementarily. MiR-142-3p target sequence can have a nucleotide sequence of SEQ ID NO:5.

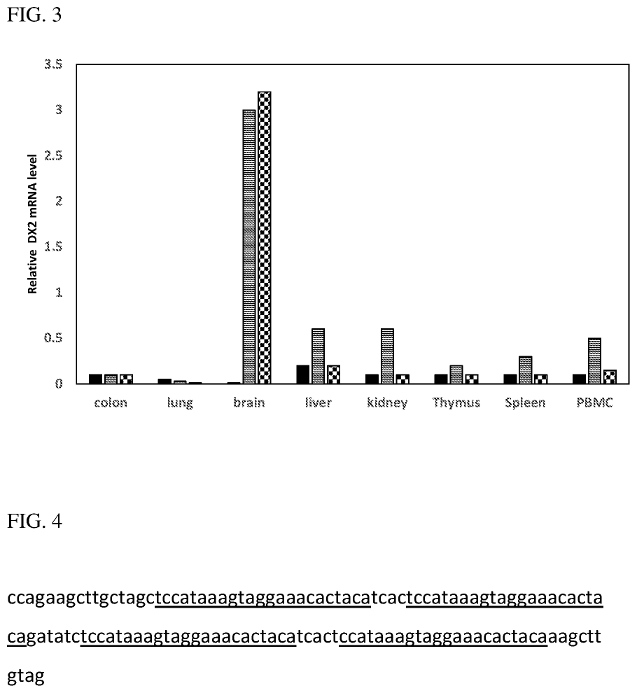

The miR-142-3p target sequence includes limiting enzyme for cloning (Nhe 1 and Hind III, Bmt 1) site sequence (ccagaagcttgctagc) and limiting enzyme (Hind H) site sequence (aagcttgtag). It includes the nucleotide sequence of SEQ ID NO:5 that has been repeated 4 times with the linkers (tcac and gatatc) that connects them (FIG. 3; SEQ ID NO:6).

### Example 1.3. Production of the Recombinant Vector

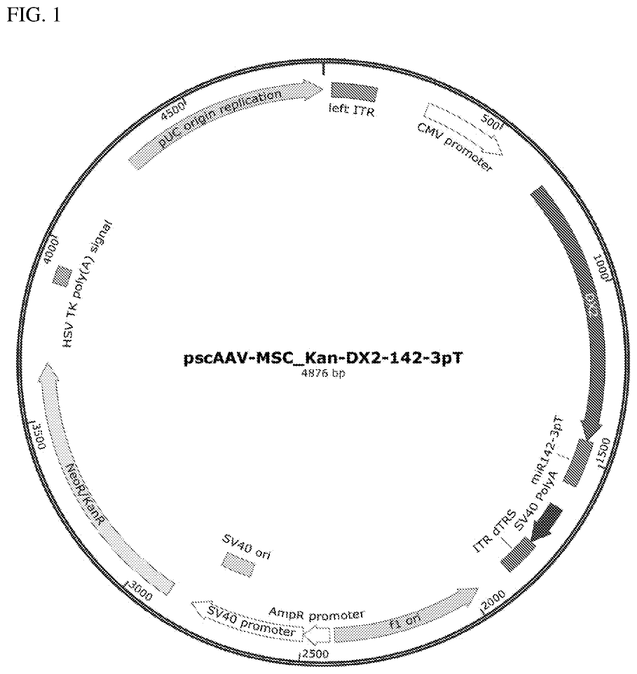

In order to produce the recombinant vector, miR-142-3p target sequence (SEQ ID NO:5) was inserted into 3′UTR of the AIMP2 variant (sequence number of 1). Connecting of the AIMP-2 variant and miR-142-3p target sequence is indicated with nucleotide sequence number of 6, and, specifically, was cut and inserted by using Nhe I and Hind III sites. The recombinant vector is shown in FIG. 1.

### Example 2. Confirmation of the Nerve Cells Specific Expression of Recombinant Vector

### Example 2.1. Confirmation of Neuron-Specific Expression Effect under In Vitro Condition

Since miR142-3p is specifically expressed only in hemopoietic cells, the extent of the expression of AIMP2 variant was confirmed in specific cells in accordance with the knockdown of AIMP2 variant according to the expression of miR142-3p target sequence of the recombinant vector.

Specifically, there were group with no treatment of the recombinant vector (SHAM), void/control vector processed group (NC vector), single AIMP2 variant vector processed group (pscAAV_DX2) and group treated with the recombinant vector (pscAAV-DX2-miR142-3pT). The concentration of all the vectors is in the unit of ug/ul and each group was treated with 2.5 ul (2.5 ug). In each of the treatment groups, treatments were made on the THP-1 cells strain (human leukemic monocyte cells) and SH-SY5Y cells strain (neuroblastoma) with confirmation of knockdown of AIMP2 variant. qPCR was executed by using the primers in the Table 1 below (degeneration for 15 seconds and annealing and extension over 40 cycles under the temperature of 60° C. for 30 seconds).

TABLE 1

| AIMP2 | | SEQ ID |
|---|---|---|
| variant | Primer | NO: |
| Forward | CTGGCCACGTGCAG | 8 |
| | GATTACGGGG | |
| | (only human) | |
| Reverse | AAGTGAATCCCAGC | 9 |
| | TGATAG (only human) | |

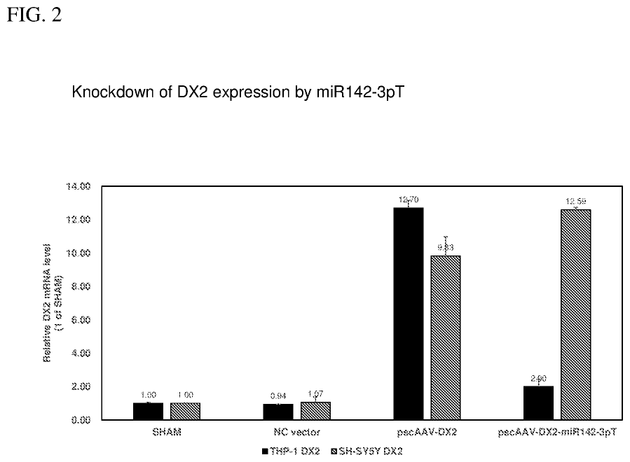

As the result, it was confirmed that AIMP2 variant is not expressed in the SHAM and NC vector groups. In addition, it was confirmed that there was expression in both the THP-1 cell strain and SH-SY5Y cell strain of the single AIMP2 variant vector processed group (pscAAV-DX2), thereby confirming that nerve cell-specific expression is not induced. On the other hand, it was confirmed that the AIMP2 variant is specifically expressed only in the SH-SY5Y cell strain in the group treated with the recombinant vector (FIG. 2).

### Example 2.2. Confirmation of Nerve Cell-Specific Expression Effect under In Vivo Conditions

Specifically, there were void/control vector processed group (NC vector), single AIMP2 variant vector treated group (pscAAV-DX2) and group treated with the recombinant vector of the invention (pscAAV-DX2-miR142-3pT). Intraparenchymal treatment with 10 ul (10⁹ vg) each of the virus with concentration of 10⁸ vg/ul was executed. After the intracranial injection of each of the treated groups into the mouse, expression of AIMP2 was confirmed in large intestinal tissues, lung tissues, cerebral tissues, hepatic tissues, renal tissues, thymus tissues, spleen tissues and peripheral blood mononuclear cells (PBMC) after 1 week. qPCR was executed by using the primers in the Table 1 below (degeneration for 15 seconds, and annealing and extension over 40 cycles under the temperature of 60° C. for 30 seconds).

As the results, it was confirmed that the expression of AIMP2 variant specifically increased only in the brain tissue with highly concentrated neurons in the group treated with the recombinant vector of the invention (FIG. 3). On the other hand, it was confirmed that the expression of AIMP2 variant is hindered in tissues other than the brain tissue.

### Example 3. Materials and Methods

### Example 3.1. qRT-PCR

Total RNA was isolated from spinal cord using TRIzol (Invitrogen, Waltham, MA, USA) according to the manufacturer's protocol. The extracted RNA was quantified by a spectrophotometer (ASP-2680, ACTgene, USA) for quantification. For making cDNA, a reverse transcription was performed using the SuperScript III First-Strand (Invitrogen) through manufacturer's protocol. The resulting cDNA was used for real-time PCR using SYBR green PCR master mix (ThermoFisher Scientific, USA). Expression data of the duplicated result were used for 2-ΔΔCt statistical analysis and GADPH expression was used for normalization.

### Example 3.2. Animals

hSOD1 G93A transgenic mice (B6.Cg-Tg(SOD1*G93A)1Gur/J) used in this study were purchased from the Jackson Laboratories (Bar Harbor, ME, USA). Age matched WT control mice were also used. The animals were housed in individual cages under specific pathogen-free conditions and a constant environment condition (21-23° C. temperature, 50-60% humidity and 12-h light/dark cycle) in the animal facility of Seoul National University, Republic of Korea. All experimental procedures were performed in accordance with guidelines of the Seoul National University Institutional Animal Care and Use Committee (SNUIACUC, Aug. 7, 2017) and this study was approved by our local ethic committee “SNUIACUC” (Approval No. SNU-170807-1). In pre-symptomatic stage, same age, female mice were administrated with AAV-GFP and DX2 vector. AAV-DX2 transduction were intrathecally injected by direct lumber puncture. Total 8 μl (4 μl/point) of AAV-GFP or DX2 vector with a Hamilton syringe (Hamilton, Switzerland) was slowly injected (1 μl/min) at two points while the needle was slowly retracted to prevent loss of injected vector.

### Example 3.3. miR142-3p Inhibition Experiment

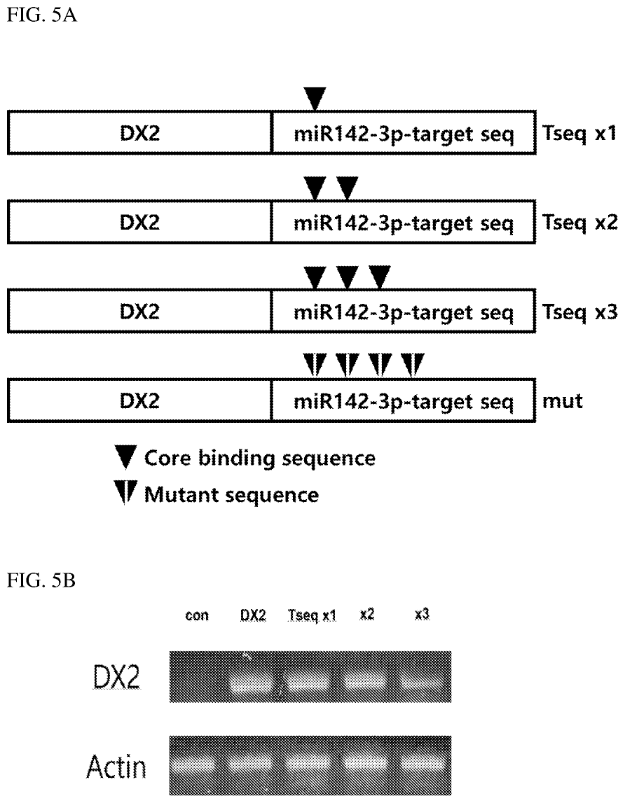

miR-142-3p inhibition on DX2 expression could be observed from ×1 miR-142-3p target sequence. The HEK293 cells were transiently transfected with the ×1, ×2, and ×3 repeat miR-142-3p target sequence vectors, and also with 100 pmol miR-142-3p using lipofectamine 2000 (Invitrogen, US), and then incubated for 48 hrs. The amount of DX2 mRNA was analyzed by PCR. miR142-3p inhibition on DX2 expression was observed from Tseq ×1 repeat miR142-3p target seq (FIG. 5B).

### Example 4

### Example 4.1. Three Types of Vectors Generated for Inhibition Effect of Core Binding Sequence

Tseq ×1 contains 1 core binding sequence, Tseq ×2 contains 2 core binding sequences, and Tseq ×3 contains 3 core binding sequences (FIG. 5A).

miR142-3p (100 pmol) inhibition on DX2 expression was started to be observed from ×1 repeat miR142-3p target sequence. The HEK293 cells were transiently transfected with the ×1, ×2, and ×3 repeat miR-142-3p T seq vectors, and also with 100 pmol miR-142-3p using lipofectamin 2000 (invitrogen, US), then incubated for 48 h. Amount of DX2 mRNA was analyzed by PCR. When the number of core binding sequence in miR142-3p target seq are increased, miR142-3p inhibition on DX2 expression was also increased. Tseq ×3 core sequence containing vector showed significant inhibition (FIG. 5B).

### Example 4.2. Core Sequence Mutation

Using mouse B cell microarray data and mirSVR score of miR-142-3p target gene, core sequence was predicted. Four regions of core sequences were substituted as follows: (5′-AACACTAC-3′→5′-CCACTGCA-3′) (see FIG. 4 for original sequence and FIG. 5A for schematic drawing).

### Example 4.3. Core Binding Sequence is Important DX2 Inhibition

Four core sequences were substituted (FIG. 5A). The HEK293 cells were transiently transfected with the DX2-miR-142-3p T seq ×3 repeated vector (Tseq3×) or with core sequence mutated vector (mut), and with 100 pmol miR-142-3p by using lipofectamin 2000 (Invitrogen, US), and then incubated for 48 hrs. Expression of DX2 mRNA was analyzed by PCR. Tseq ×3 repeated vector which showed significant inhibition of DX2 (FIG. 5B) and DX2 construct were used as control. 100 pmol of miR142-3p treatment inhibited Tseq ×3 vector significantly but DX2 and mut sequence were not inhibited (FIG. 6).

### Example 4.4. Tissue Distribution Data in ALS Mouse Model

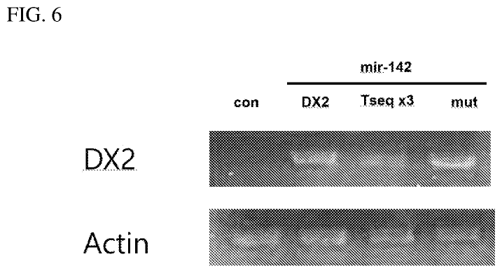

Total RNA from the spinal cord was extracted following intrathecal injection of the scAAV2-DX2-miR142-3p. qRT-PCR was performed. DX2 expression should be limited only in the local injection site, the spinal cord. hSOD1 G93A transgenic mice, scAAV-DX2 miR142-3p was expressed with intrathecal injection. Control vehicle injection showed expression only in spinal cord, not brain nor sciatic nerve (FIG. 7).

### Example 5

In Example 2, HEK293T cells were co-transfected with the three plasmids from Oxgene, UK, that encode all the components necessary to produce recombinant AAV2 particles.

HEK293T cells were also transfected with only pSF-AAV-ITR-CMV-EGFP-ITR-KanR (Oxgene, UK) with an insertion of AIMP2-DX2 or DX2-miR142 target nucleotide as expression vectors and not for producing AAV particles.

DX2 coding vector (2 ug) and DX2-miR142 target seq coding vector (2 ug) were transfected into THP-1 cell (human monocyte, CD45+ cell) and SH-SY5Y (neuronal cell). After 48 hrs, the cells were harvested and mRNA was isolated. With the synthesized cDNA, the expression of DX2 was analyzed by real-time PCR.

Whereas DX2 expression level was similar between DX2 coding vector and DX2-miR142 target seq coding vector transfected SH-SY5Y, DX2 expression was dramatically decreased in DX2-miR142 target seq coding vector transfected THP-1 cells. Thus, miR142-3p worked only in THP-1 cells (FIG. 8).

### Example 6

### Example 6.1. Experimental Methods

### Animal Models

hSOD1^{G93A} transgenic mice (B6.Cg-Tg(SOD1*G93A)1Gur/J) used in this study were purchased from the Jackson Laboratories (Bar Harbor, ME, USA). The animals were housed in individual cages under specific pathogen-free conditions and a constant environment condition (21-23° C. temperature, 50-60% humidity and 12-h light/dark cycle). In pre-symptomatic stage, same age, female mice were administrated with AAV2-GFP or AAV2-DX2. AAV2-DX2 transduction were intrathecal injected by direct lumber puncture. Total 8 μl (4 μl/point) of AAV-GFP or DX2 vector with a Hamilton syringe (Hamilton, Switzerland) was slowly injected (1 μl/min) at two points while the needle was slowly retracted to prevent loss of injected virus.

Behavioral analysis To determine the onset of disease, we pointed out the day that the mice started to lose body weight up to 5-6% from maximum body weight. In general, severe symptomatic stages is known to be observed from 12 weeks after birth in SOD1G93A mice, but motor performance deficits began several weeks prior to the onset of overt symptoms (postnatal day 45) (C. R. Hayworth et al. Neuroscience. 2009 Dec. 15; 164(3): 975-985). In the present study, at 9 weeks after birth when limping is observed and the mice started to lose up to 5-6% of their maximum body weight, scAAV-GFP or scAAV-DX2 (GO102) was administered to same age, female mice. AAV2-DX2 (GO102) transduction was achieved via intrathecal injection by direct lumbar puncture.

### Example 6.2. Results

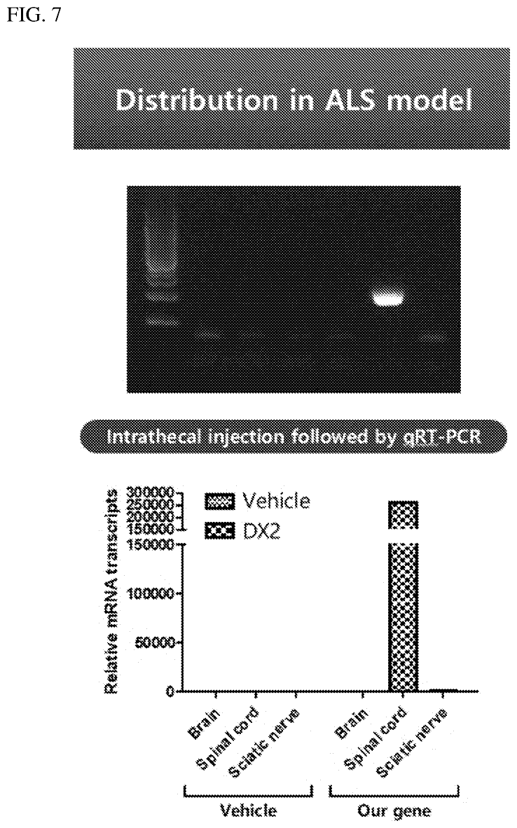

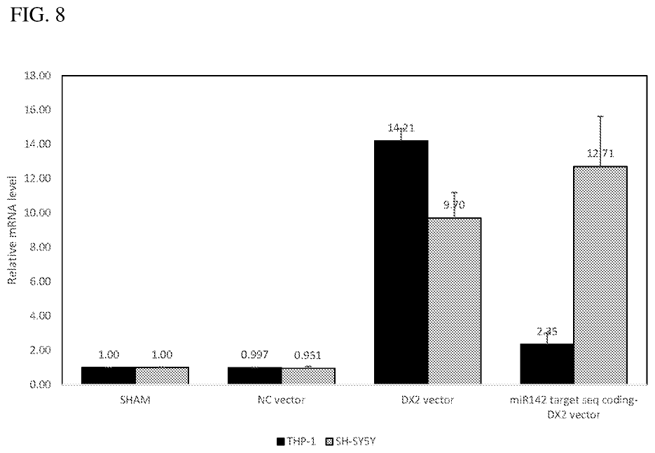

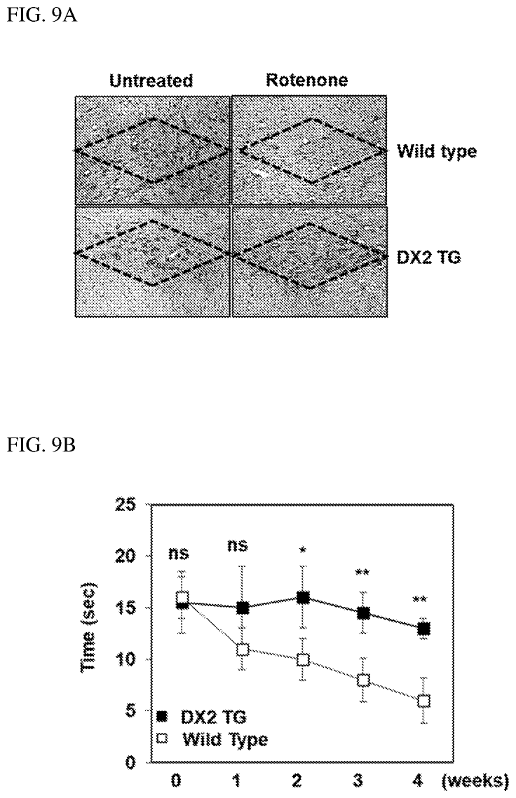

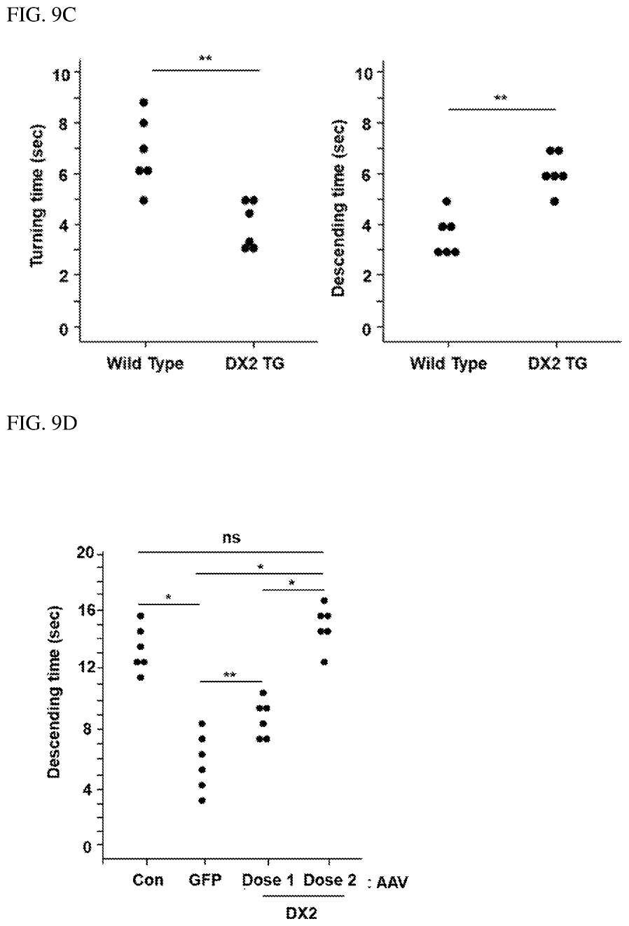

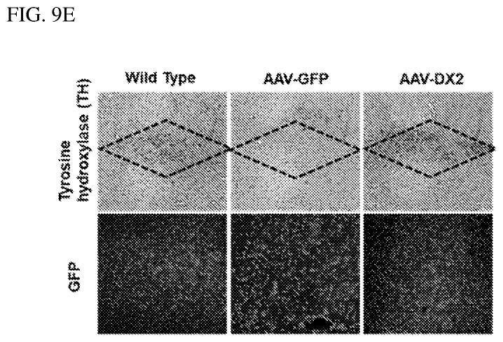

FIG. 9A-9C shows that DX2 transgenic mice recover motor symptoms in rotenone-treated mice. FIG. 9A shows that TH expression was analyzed with mice brain in the indicated mice. The black dotted square shows TF-stained regions. FIG. 9B shows a Rotarod analysis. Latency to fall in rotenone-treated wild type and DX2 transgenic (TG) mice. FIG. 9C shows a Pole test. Vertical movement (left panel) and T-turn time (right panel) in rotenone-treated wild type and DX2 TG mice. Animals; n=6 (in each group), ns; non-significant, **P<0.01, *P<0.05, t-test. FIGS. 9D and 9E show that DX2 improves neuronal damage and behavior in rotenone-induced PD mouse model. FIG. 9D shows a pole test. scAAV-DX2 recovered motor coordination and balance in the rotenone-treated PD mouse model. “Con” and “GFP” indicate wild type and rotenone-treated GFP injection mice. “Dose 1” and “Dose 2” represent the different injection dose of DX2 in rotenone-treated mice. FIG. 9E shows immunohistochemistry and immunofluorescence image of the mouse substantia nigra. The upper panel shows TH-positive cells in the striatum and the lower panel indicates the distribution of an injected-GFP expressing virus. The black dotted square indicates the stained region of TH. Animals; n=5 (in each group), ns; non-significant, **P<0.05, *P<0.01, t-test.

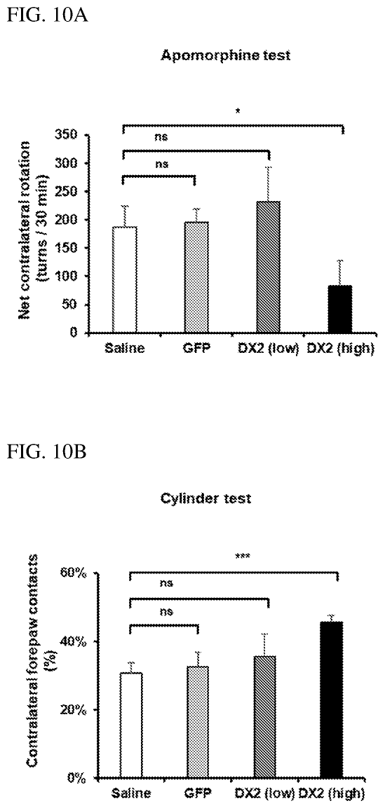

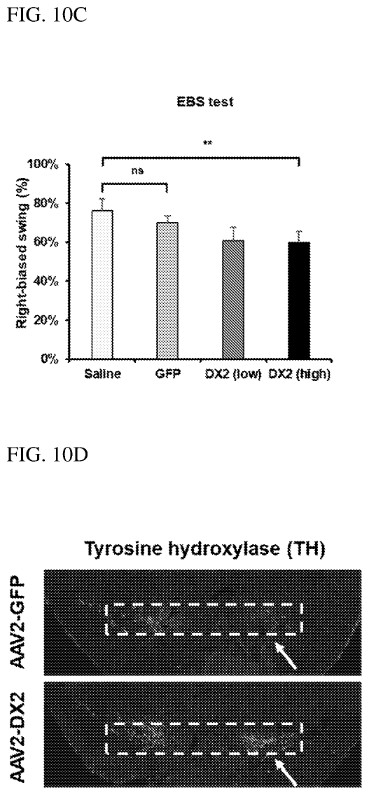

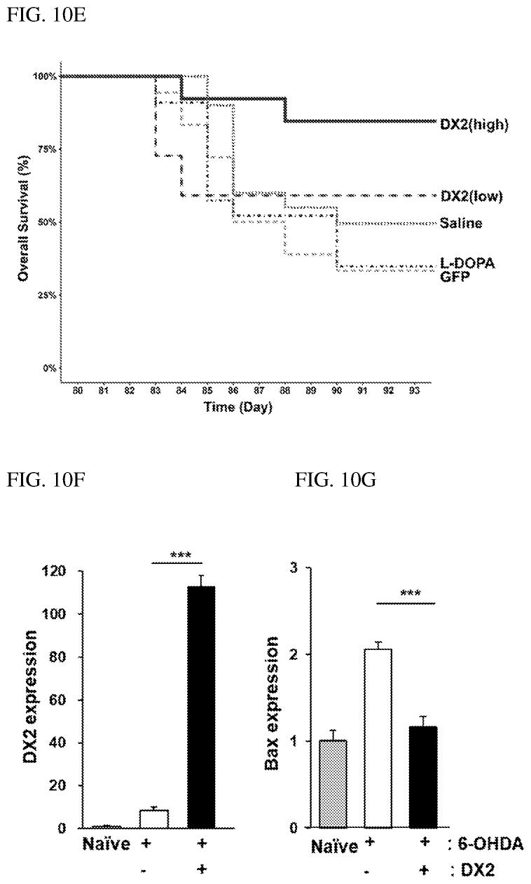

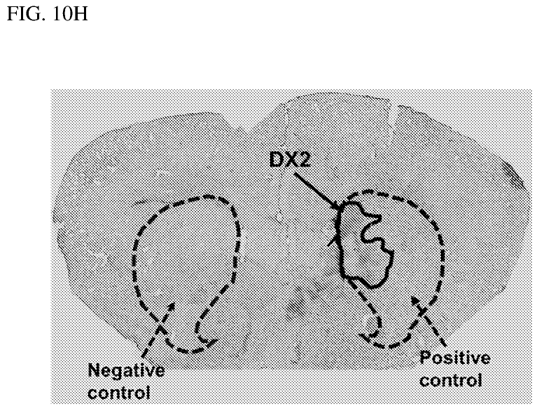

FIG. 10A-10H show that DX2 prevents behavioral deficits in the 6-OHDA-induced PD model. FIG. 10A demonstrates that scAAV-DX2-treated mouse showed lower levels of contralateral rotation compared to that of saline or vehicle (GFP), indicating that DX2 attenuated damage in dopaminergic neurons. FIG. 10B demonstrates that DX2-treated mice showed increased contralateral forepaw contacts, indicating that AAV-DX2 attenuated unilateral damage in dopaminergic neurons. FIG. 10C demonstrates that AAV-DX2 treated mouse showed less right-biased body swing. Animals; saline (saline-treated wild type mice) n=4, GFP (GFP-injected 6-OHDA-treated mice) n=5, DX2 (DX2-injected 6-OHDA-treated mice) n=11, scAAV; scAAV-GFP 4×10⁹ vg, scAAV-DX2 4×10⁹ vg, ns; non-significant, *P<0.05, **P<0.005, ***P<0.001, t-test. FIG. 10D shows immunofluorescence image of GFP and DX2-injected mice brain. The white square box indicates TH positive dopaminergic neuronal cells and the white arrows shows indicated virus injection site. FIG. 10E shows the survival rate in each mice group. Animals; n=15, Saline indicates saline-treated wild type mice. L-DOPA, GFP, and DX2 represent L-DOPA, GFP, and DX2 injection in 6-OHDA-treated mice. scAAV; scAAV-GFP (GFP) 4×10⁹ vg, scAAV-DX2 (DX2) (low) 1.6×10⁸ vg, scAAV-DX2 (DX2) (high) 4×10⁹ vg. FIGS. 10F and 10G show DX2 and Bax mRNA expression of naïve, 6-OHDA and DX2-treated mice. ***P<0.001, t-test. FIG. 10H shows RNA in situ hybridization to identify the DX2 expressed cells in AAV-DX2 injected 6-OHDA mice model.

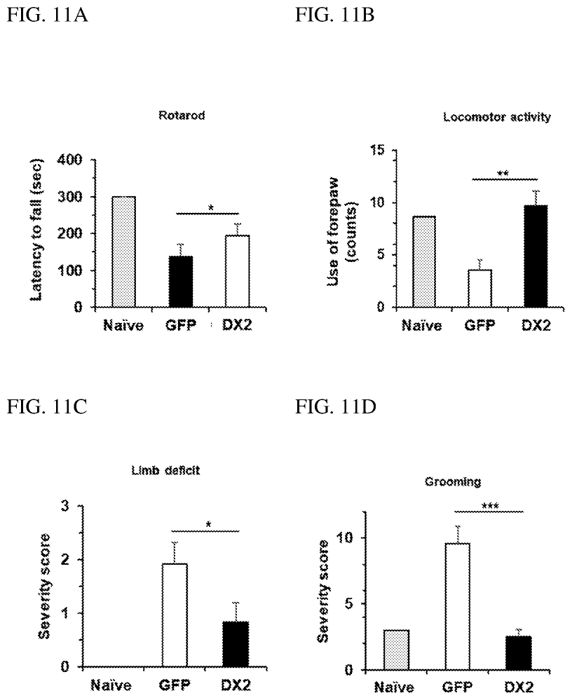

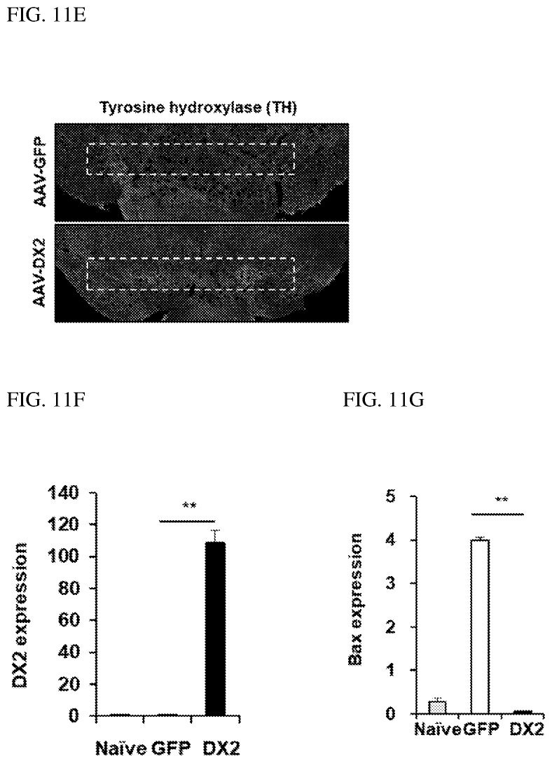

FIGS. 11A-11G show that DX2 restores motor symptoms in MPTP-induced PD model. FIG. 11A demonstrates scAAV-DX2-treated mouse showed slightly longer latency to fall in the rotarod test when compared with that of vehicle (scAAV-GFP, GFP) indicating that scAAV-DX2 attenuated damage towards dopaminergic neurons. FIG. 11B demonstrates that DX2-treated mouse showed improved locomotor activity based on the SHIRPA test. FIG. 11C demonstrates that DX2-treated mice showed a relatively lower level of limb deficit. FIG. 11D demonstrates that DX2-overexpressed mouse showed improved grooming rate when compared with vehicle control (GFP). FIG. 11E shows that immunofluorescence image of TH-positive cells in the mouse substantia nigra. The white square box indicates the TH expressing regions. FIGS. 11F and 11G show DX2 (FIG. 11F) and Bax (FIG. 11G) mRNA expression of the indicated mice brain. Naïve, GFP, and DX2 indicate saline-treated wild type mice, GFP-injected MPTP-treated mice, and DX2-injected MPTP-treated mice. Animals; Naive n=6, GFP n=9, DX2 n=12, scAAV; scAAV-GFP 4×10⁹ vg, scAAV-DX2 4×10⁹ vg, *P<0.05, **P<0.001, ***P<0.0001, t-test.

### Example 7

SOD1 transgenic mice were treated with AAV-GFP (GFP) or AAV-DX2 in the spinal canal to explore the effects of DX2 in vivo. The onset of the disease was delayed in the DX2-injected mice group compared to the GFP-injected mice group. Moreover, mice in the group in which DX2 was administrated survived significantly longer than those in the GFP injected group. The lifespan of the DX2-administrated mice was prolonged compared to the GFP-injected mice.

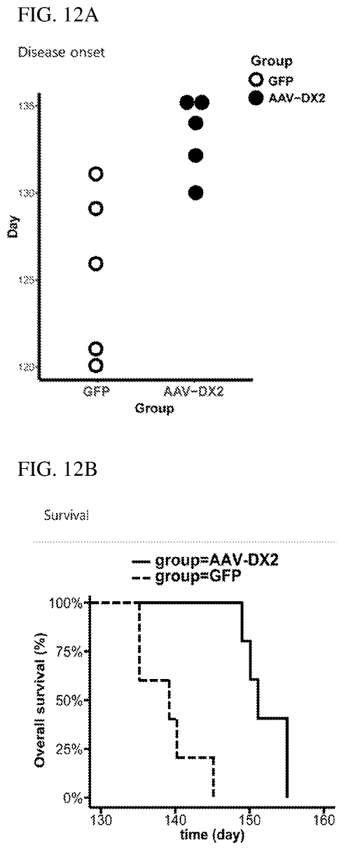

FIGS. 12A and 12B show that administration of DX2 improves the disease onset and prolongs the lifespan of mice in Lou Gehrig's disease model. FIG. 12A. Disease onset was improved in AAV-DX2 group. FIG. 12B. The lifespan of mice was prolonged in the AAV-DX2 group when compared to those in the AAV-GFP group. Animals; n=5.

### Example 8

### Example 8.1. Experimental Methods

### Cell Culture and Treatment

SK-SY5Y cells, human neuroblastoma cell lines, were maintained in RPMI 1640 containing 10% fetal bovine serum, 100 unit/ml penicillin and 100 μg/m1 streptomycin. For the induction of Alzheimer's disease (AD) in neuronal cells, SK-SY5Y cells were seed on 6 well plates at a density of 1×10⁶ cells/well, and 16 hours later, the culture media were replaced with RPMI 1640 containing 25 μM amyloid β-protein oligomer (Aβ-O) for 24 hours. To identify the inhibitory effect of neuronal cell death by DX2 expression, SK-SY5Y cells were incubated with Aβ-O for 24 hours and then, vehicle (scAAV2-GFP) or overexpressed-DX2 (scAAV2-DX2) virus was used to treat cells for 48 hours in RPMI 1640 growth media. Cell death was analyzed by western blot and microscopy.

### Immunoblot Analysis

SH-SY5Y cells were lysed in 25 mM Tris-HCl, pH 7.4 containing 150 mM NaCl, 0.5% Triton X-100 and protease inhibitor cocktail. Samples containing 50 μg of protein were blotted in 10% polyacrylamide gel and electrophoretically transferred onto membrane. The membrane was blocked with 5% non-fat dry milk in Tris-buffered saline with 20% Tween-20 and incubated with primary antibodies against p53 and actin. The antibodies on membrane were detected with horseradish peroxidase-conjugated secondary mouse anti-goat and anti-rabbit antibodies. The membrane was analyzed by SuperSignal West Dura extended-duration substrate according to manufacturer's manual (Thermo Fisher Scientific, Waltham, MA, USA).

### Example 8.2. Results

### AIMP2-DX2 Attenuates Aβ-O-Induced Neuronal Cell Death

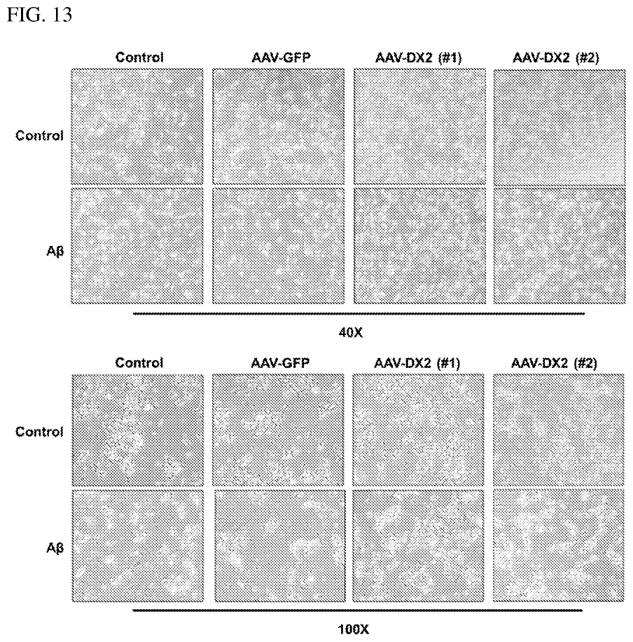

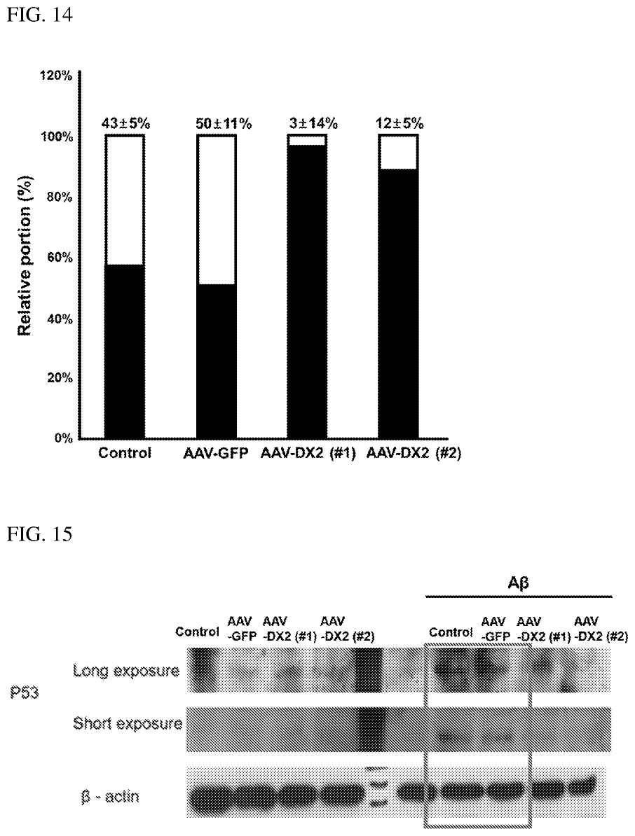

Alzheimer's disease (AD) is a progressive neurodegenerative disease that is caused by the accumulation of abnormal protein, such as amyloid β-protein (Aβ) and phosphorylated tau (p-tau) protein, in the brain (Duyckaerts 2009). It is known that amyloid β-protein aggregation by proteolytic cleavage of the amyloid precursor protein play a critical role in AD development (Viola 2015 and De Strooper 2010). Thus, we studied whether overexpression of AIMP2-DX2 (DX2), an inhibitory factor of cell death (Choi 2011), in AD-induced cells may affect neuronal cell death. In FIG. 13, the cell survival was not different in untreated, vehicle-treated (AAV-GFP) and DX2-treated (AAV-DX2) cells at normal growth, suggesting that increased DX2 expression in normal condition is not cause of neuronal cell survival. In Aβ-O treatment condition, decreased neuronal cell death was observed in DX2-treated cells (Aβ+AAV-DX2) compare to vehicle-treated cells (Aβ+AAV-GFP). And the neuronal cell death in FIG. 14 was quantitatively analyzed and the percentage of cells was scored after observing cells in three different field of FIG. 13. As shown FIG. 14, DX2 overexpressing cells have significantly increased neuronal cell viability compare to vehicle-treated group (up to 47%). These results indicate that DX2 expression is important factor for the protective effect of Aβ-O-induced cell death.

### DX2 Inhibits Aβ-O-Induced p53 Expression

P53, tumor suppressor protein, is a key factor, which is regulated biological events such as cell cycle and apoptosis (Finlay 1989). As shown in previous reports (Choi 2011), AIMP2 binds to the N-terminal of p53, which is binding domain for Mdm2 and its binding induces the stability of p53 and pro-apoptotic activity. Also, it is known that DX2 inhibits the apoptotic activity of AIMP2 by interrupting interaction with p53. Thus, it was studied whether increased expression of DX2 by viral transduction of DX2 gene affects p53 expression in Aβ-O-treated neuronal cells. In FIG. 15, the cellular expression level of p53 was not altered in normal growth condition, but the expression level of p53 was increased in the presence of Aβ-O. Also, in DX2-treated cells, DX2 expression decreased Aβ-O-induced p53 expression. These results suggest that DX2 inhibits Aβ-O-induced apoptosis and pro-apoptotic protein expression, such as p53, in neuronal cells.

DX2 expression plays an important role in neurotoxin-induced p53 expression (FIG. 15). SK-SY5Y cells were incubated with AAV-DX2 or AAV-GFP in the absence or presence of 25 μM Aβ-O. After 48 hours, total protein lysates were prepared, and the level of p53 protein was analyzed by immunoblot analysis. The level of β-actin was analyzed as a loading control. The red square box indicates increased level of p53 in Aβ-O-treated cells.

### Example 9

### Example 9.1. Materials and Methods

### Cell Culture and Reagent

HEK 293 cell line was obtained from American Type Culture Collection (ATCC, Manassas, VA, USA) and Neuro-2A (N2A), SK-N-SH and SH-SY5Y cells were obtained from Korean Cell Line Bank (KCLB, Seoul, KOREA). HEK 293 cells and N2A cells were grown in Dulbecco's modified Eagle's medium supplemented with 10% fetal bovine serum (FBS) and 1% penicillin-streptomycin (HyClone, Pittsburgh, PA, USA). And SK-N-SH cells were incubated in RPMI-1640 with 10% FBS and 1% antibiotics. The transient transfection of myc-tagged KARS, HA-tagged mutant SOD1, GFP-tagged KARS, and GFP-tagged mutant SOD1 were transfected by lipofectamine 2000 (Invitrogen, Carlsbad, CA, USA). And 3-(4,5-dimethylthiazol-2-yl)-2,5-diphenyltetra-zolium bromide (MTT) and HEMA (2-hydroxyethyl methacrylate) were from Sigma-Aldrich (St. Louis, MO, USA).

### Yeast-Two Hybrid Assay

Full length KARS and fragmented KARS were cloned into pLexA plasmid and SOD1 WT, SOD1 G85R, and SOD1 G93A were cloned into pB42 plasmid. The positive interaction between LexA-fragments of KARS and B42-SODWT/SOD85/SOD93 in yeast was determined by LEU2 and LacZ reporter system using X-gal plate (21).

### Immunoprecipitation Assay

Cell lysates were harvested and prepared by RIPA buffer (50 mM Tris-HCl pH 8.0, 1 mM EDTA, 150 mM NaCl, 20% glycerol, 1% NP-40, 0.5% sodium deoxycholate, and PMSF). Cell lysates were incubated for 30 minutes on ice followed by collecting supernatant after centrifugation for 10 minute at 12,000 g. Anti-HA or anti-Myc agarose beads were added to lysates and incubated overnight at 4 degrees with a rocking platform. Agarose beads bounded proteins were washed three times and collected samples were separated via SDS-PAGE and western blotting analysis was performed.

### Western Blotting and Antibodies

The cells were lysed in 25 mM Tris-HCl, pH 7.4 containing 150 mM NaCl, 0.5% Triton X-100 and protease inhibitor cocktail. Samples containing 50 μg of protein were blotted in 10% polyacrylamide gel and electrophoretically transferred onto membrane. The membrane was blocked with 5% non-fat dry milk in Tris-buffered saline with Tween-20 and incubated with primary antibodies against Myc (Santa Cruz biotechnology, sc-40), HA, GFP, 67 laminin receptor, IκB, Tubulin, β-actin, TRAF2, EPRS, KARS, AIMP2, Erk, phosphorylated Erk. The antibodies on membrane were detected with horseradish peroxidase-conjugated secondary mouse anti-goat and anti-rabbit antibodies. The membrane was analyzed by SuperSignal West Dura extended-duration substrate according to manufacturer's manual (Thermo Fisher Scientific, Waltham, MA, USA).

### Immunocytochemistry

Cells were fixed with 4% PFA at room temperature for 10 min followed by wash with PBS and incubated overnight with antibodies against SOD1, 67LR. And stained-cells were washed followed by incubation with Alexa Fluor-linked IgG (Vector Laboratories INC, Burlingame, CA, USA). Nuclear DNA was stained with DAPI (4′, 6-diamidino-2-phenylindole, Thermo Fisher Scientific, Waltham, MA, USA).

### Cell Migration Assay

Migration assay was performed using 8 μm Transwell chamber (Corning INC, Corning, NY, USA). N2A cells in serum free media were seeded on the upper chamber of 24 well migration plate. The lower chamber was filled with 400 μL of DMEM with 10% FBS. After 24 hours, upper chamber was fixed with 10% PFA for 10 min at room temperature followed by staining with crystal violet. And then, migrated cells were counted.

### Cell Viability Assay

For MTT assay, 5×10⁴ cells/well were plated on 96-well plate and were treated for 24 h with specified molecule. After appropriate incubation, 15 μL of 5 mg/mL MTT solution in PBS (pH 7.2) was added in each well and incubated for 4 h at 37° C. in 5% CO₂ atmosphere. The solution was removed and dimethyl sulfoxide (DMSO) was added in each well to dissolve insoluble formazan precipitate and the absorbance was measured at 620 nm by plate reader.

### Subcellular Fractionation

To determine cellular localization of KARS1, cytosolic and membrane fractions were collected using subcellular fraction kit (Biovision, Milpitas, CA, USA). Briefly, the cells were lysed and centrifuged at 1,000 rpm for 10 min at 4° C., and the supernatant was used as the cytosolic fraction. Then, the pellets were washed and incubated with sodium deoxycholate buffer at 4° C. for 10 min and used as the membrane fraction.

### Attachment Strength Test of Cells

SOD1 G93A and DX2 transfected SH-SY5Y cells were seeded (1.0×10⁴ cells/mL) to 96 well e-plate (ACEA Biosciences, San Diego, CA, USA) and treated with TNF-α for 24 h to screen for cell adhesion. And then, attached cells were counted by iCELLigence (ACEA Biosciences, San Diego, CA, USA).

### Example 9.2. Results

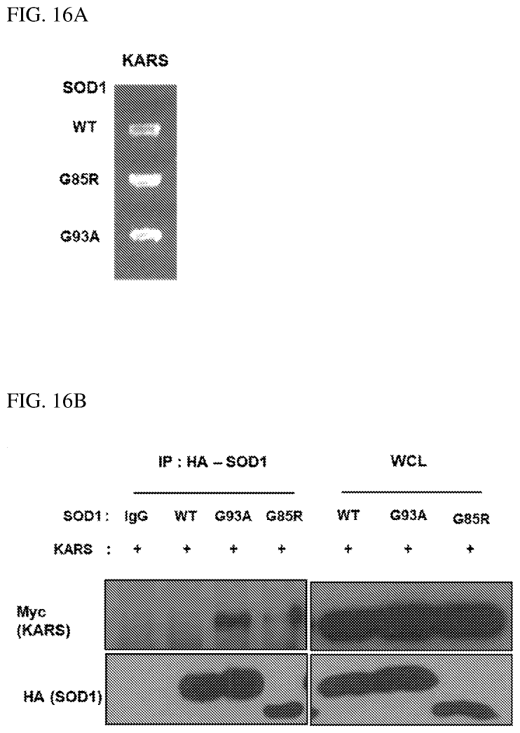

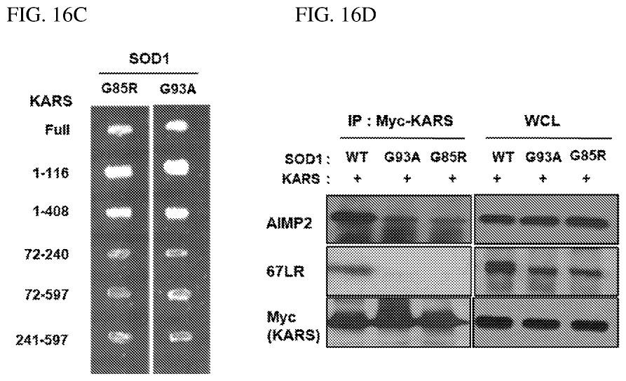

It was previously reported that mitochondrial form of KARS interacted with mutant forms SOD1 and mutant SOD1 and mitoKARS result in mitochondrial morphological abnormalities and cell toxicity. Therefore, to investigate whether KARS can regulate neuronal cell death by SOD1 mutations, we first confirmed the binding efficiency of mutant SOD1 and KARS. For the experiments, WT SOD1, SOD1 G93A, SOD1 G85R and KARS were prepared and interaction between KARS and each SOD1 was analyzed by yeast two hybrid (FIG. 16A) and immunoprecipitation analysis (FIG. 16B), and we observed that KARS binds to mutant SOD1 with much stronger binding than WT SOD1 (FIGS. 16A and -16B).

Next, to study the specific binding site of KARS and mutant SOD1, we confirmed the interaction of truncated KARS and mutant SOD1 using yeast two hybrid assay system. As shown in FIG. 16C, KARS and mutant SOD1 binding was observed at the N-terminal of KARS. It is shown that AIMP2 and 67 laminin receptor interacts with N-terminal of KRS for cancer cell migration and regulation of cell survival. Since AIMP2, 67 LR and mutant SOD1 bind to the N-terminal of KARS, we investigated whether the interaction of KARS and mutant SOD1 affects the binding of KARS to AIMP2 and 67LR. As shown in FIG. 16D, AIMP2 and 67LR bound KARS in the presence of WT SOD1, however, reduced binding of KARS to AIMP2 and 67LR was observed in the presence of mutant SOD1. The results showed that mutant SOD1 decreases binding of KARS to AIMP2 and 67LR through the binding competition of N-terminal of KARS.

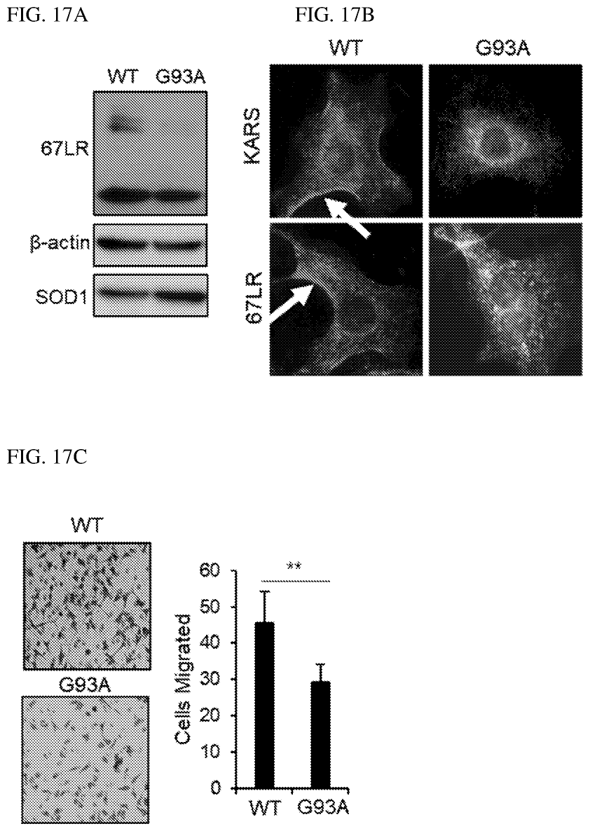

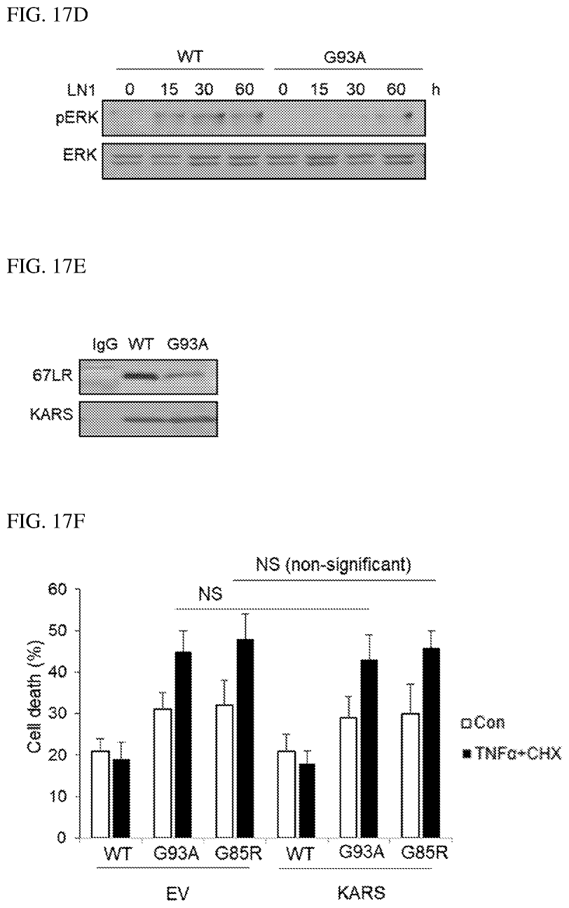

Since we showed that mutant SOD1 G93A had the best binding to KARS, we wanted to investigate its effect on 67LR and explore whether it was correlated to neural cell death. When we transfected mutant SOD1 to SK-N-SH cells, we could find that the level of 67LR was decreased (FIG. 17A).

To confirm the location of expression of 67LR we performed IF (immunofluorescence) in the mutant SOD1 transfected cells. It was shown that the KARS level is more concentrated in the cytoplasm than the membrane and is highly decreased from the membrane region (FIG. 17B).

It was previous shown that KARS induced the migration of cells through 67LR. When the cells were transfected with SOD1 wild type or mutant SOD1, the cell migration was suppressed with mutant SOD1 compared to wild type SOD1 (FIG. 17C).

Since mutant SOD1 has an effect on expression of 67LR, we explored its effect on the signaling pathway of laminin and we could confirm that mutant SOD1 highly reduces the pERK activity (FIG. 17D).

We also investigated whether the expression of mutant SOD1 affect the binding affinity between KARS and 67LR. In FIG. 17E, we observed reduced interaction with KARS and 67LR by mutant SOD1 expression.

Anoikis is a kind of apoptosis triggered by loss of contact between extracellular matrix (ECM) and cellular membrane protein and resistance of anoikis plays an important role in cell survival. And to induce anoikis, cells were co-transfected with mutant SOD1 and KARS and incubated with or without TNF-alpha/CHX in suspension condition. As a result, we observed that cell death was not restored by overexpression of KARS (FIG. 17F). This result suggested that regulation of cell death by laminin receptors is due to increased downstream signaling through the interaction of laminin receptor and ECM.

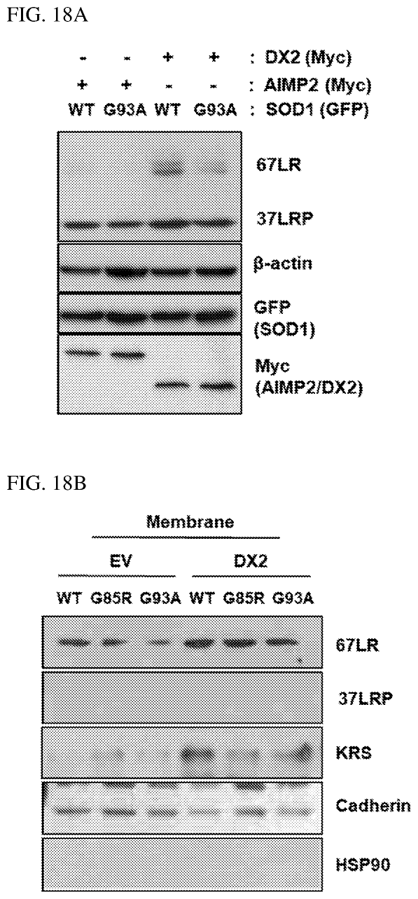

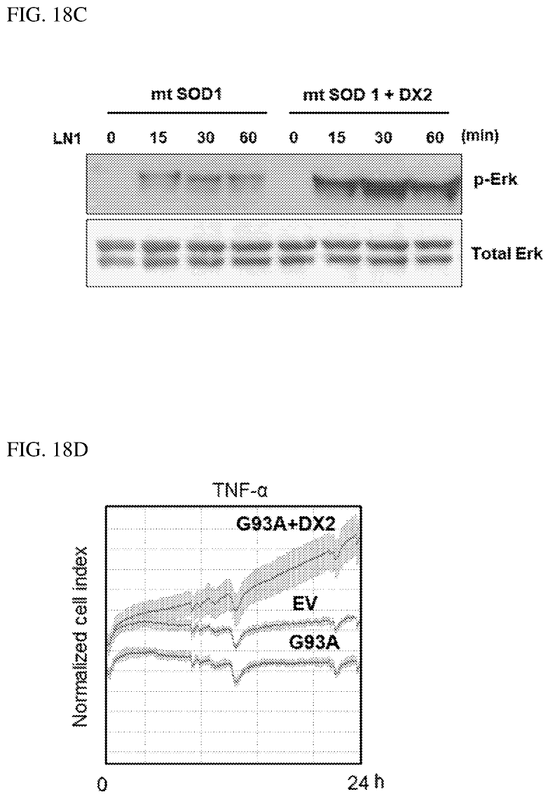

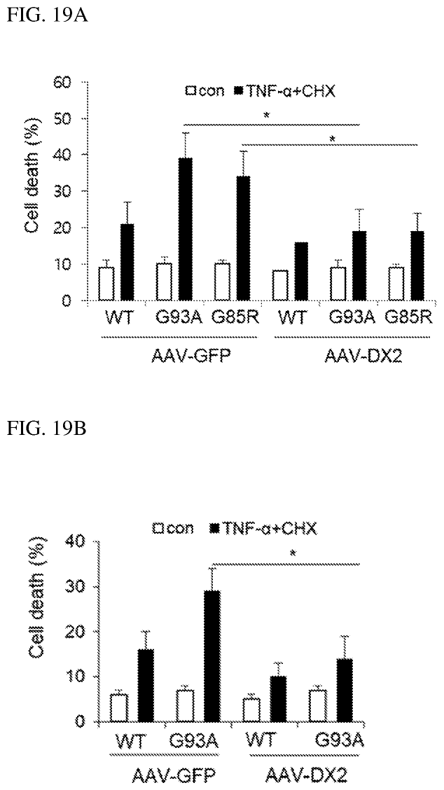

We tested whether DX2 is an important role for mutant SOD1-induced 67 LR expression. SK-N-SH cells were transfected in SOD1 WT and SOD1 G93A mutant genes and then, one group was transfected with pro-apoptotic AIMP2 genes and the other group with anti-apoptotic DX2 genes. In the presence of DX2, we observed that reduction of 67LR protein by overexpression of AIMP2 was restored (FIG. 18A).

Then, we confirmed whether the expression of 67LR reduced in plasma membrane by mutant SOD1 was restored by DX2 gene. Overexpression of DX2 in cells expressing mutant SOD1 increased 67LR protein in the cellular membrane (FIG. 18B) and we also observed that the downstream signal of 67LR was recovered by DX2 gene introduction (FIG. 18C).

Next, we tested the detachment of cells after treatment with TNF-α followed by transfection of EV, mutant SOD1 and mutant SOD1+DX2. Treatment of DX2 prevented the detachment of cells and anoikis (FIG. 18D).

We confirmed the effect of DX2 on the neuronal cell death by mutant SOD1. When AAV-DX2 was transduced in WT SOD1 or mutant SOD1 overexpressing cells, we observed that the apoptosis induced by mutant SOD1 was reduced to control level (FIG. 19A). Cell death rate of WT and two mutants, G85R and G93A, induced by CHX/TNF-α treatment were increased in GFP infected cells about 20%, respectively. However, the cell death rate by CHX/TNF-α treatment in DX2 infected cells were about 20% lower than their cell death rate of GFP-transduced cells with a significant difference (p<0.001).

And the decrease of CHX/TNF-α-induced cell death by DX2 overexpression was also shown in primary neuron. DX2 overexpressing AAV was infected in the primary neural cells extracted from wild type or SOD1 transgenic mice, transfected cells were treated with CHX/TNF-α and the cell death rate was analyzed. It was shown that G93A primary neural cells were increased cell death in CHX/TNF-α treated condition, while DX2 greatly reduced the cells death in CHX/TNF-α-treated WT and G93A primary neural cells (FIG. 19B).

### Example 10

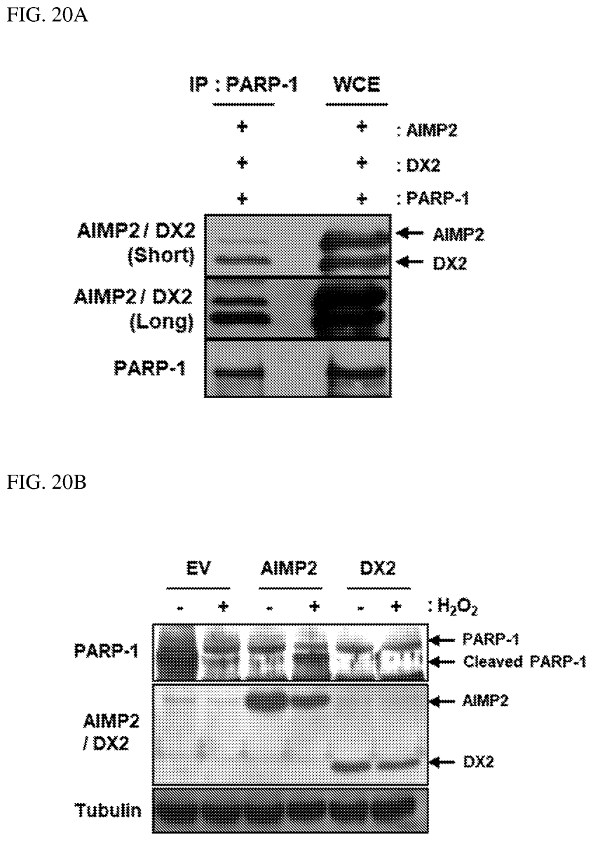

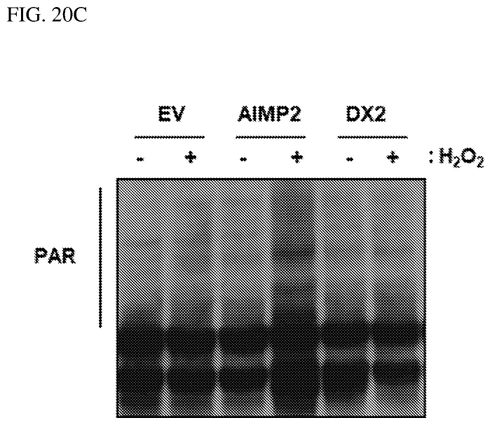

In a previous study, it was shown that AIMP2 acts as a substrate of parkin and interacts with PARP-1, and this interaction regulates neuronal cell death in PD (Lee 2013). Thus, to investigate whether DX2 is a competitive inhibitor of AIMP2 and regulates neuronal cell death, we first performed a binding assay between PARP-1 and AIMP2 or DX2. PARP-1, AIMP2, and DX2 expression was induced by transfection of each plasmid in SH-SY5Y cells and then followed by analyses of PARP-1 pull-down assays (FIG. 20A). Cells were transfected with the EV (empty vector), AIMP2, and DX2, and 24 hours later, transfected cells were incubated with 10 μM H₂O₂ for 4 hours. Cleaved PARP-1 levels (FIG. 20B) and PARlyation (FIG. 20C) were examined using immunoblot assays. In oxidative stressed-induce cellular damage conditions, DX2 attenuates cleavage of PARP-1 (FIG. 20B) and PARylation (FIG. 20C) related to cell death.

As shown in FIG. 20A, we found that DX2 binds to PARP-1 more strongly than AIMP2. To assess whether AIMP2 and DX2 can affect PARP-1 cleavage under oxidative stress conditions, we transfected them with the vector expressing empty control (EV), AIMP2 or DX2 and then, treated with hydrogen peroxide. AIMP2-transfected cells showed significantly increased cleavage of PARP-1 when compared to the expression seen in other transfected cells under oxidative stress conditions. However, PARP-1 cleavage was not observed in DX2-transfected cells (FIG. 20B).

PARylation is a post-translational process, regulating biological events such as DNA damage response and apoptosis (Szabo 1996 and Virag (1998). PARP-1 is an enzyme that recognizes damaged DNA in the nucleus, forms PAR chains, and induces degradation of damaged proteins through the PARylation. Because PARlylation, i.e. the formation of PAR polymers requires the catalytic activity of cleaved PARP-1 (Barkauskaite 2015), we investigated the effects of AIMP2 or DX2 on PARylation. As shown in FIG. 20C, the PARylation of AIMP2 was increased in the presence of H₂O₂, but the PARlylation of DX2 was not altered. Based on these results, we conclude that DX2 is an inhibitory molecule of oxidative stress-induced PARP-1 cleavage.

### Example 11

### DX2 Inhibition of Neuromuscular Junction Damage

The motor neurons are essential for the communication between the brain and the muscles and transmit vital instructions for mobility. When these nerve cells are dysfunctional or damaged, they gradually stop communicating with the muscles, and the brain loses its ability to control and initiate voluntary movements. This results in a progressive weakness, muscle twitches (fasciculations), and atrophy of voluntary skeletal muscles throughout the body. In addition, the degeneration of NMJ, leading to skeletal muscle denervation, is thought to play an essential role in the onset of ALS. Muscle twitching/fasciculation and respiratory failure typically happen in ALS within 2-3 years from the onset. In the final stages of the disease, this leads to fatal paralysis and death due to respiratory failure.

The Muscles were fixed in 4% PFA overnight at 4° C. The Muscles were dehydrated at 30% sucrose and embedded with the OCT compound for tissue cryosection. All muscle cryosection samples were acquired from the neuromuscular junction containing section with 20 μm thickness.

20 μm thick cryosections were washed twice (5 min each) in 1×PBS then incubated in a blocking solution (5% BSA) for 1 h at room temperature.

Aspirate BSA, Sections were incubated overnight with primary antibodies against the neurofliaments (stained green using anti-neurofilament plus anti-2H3, SV2) and the postsynaptic acetylcholine receptors_AChRs (stained red using fluorescent α-bungarotoxin conjugates) in blocking solution at room temperature. A number of defects can be readily observed, including partially innervated or completely denervated postsynaptic receptor sites, fragmented or shrunken postsynaptic receptors, atrophied axons or terminals, and swollen or dystrophic axons or terminals.

Immunofluorescence ROI set and overlapping coefficient measurements were measured with Image J.

On this basis, skeletal muscle denervation in each of wildtype (WT), ALS induced model (AAV-GFP), and ALS induced model (GO102) groups were measured by double staining of the gastrocnemius muscles for alpha-bungarotoxin and SV2, 2H3.

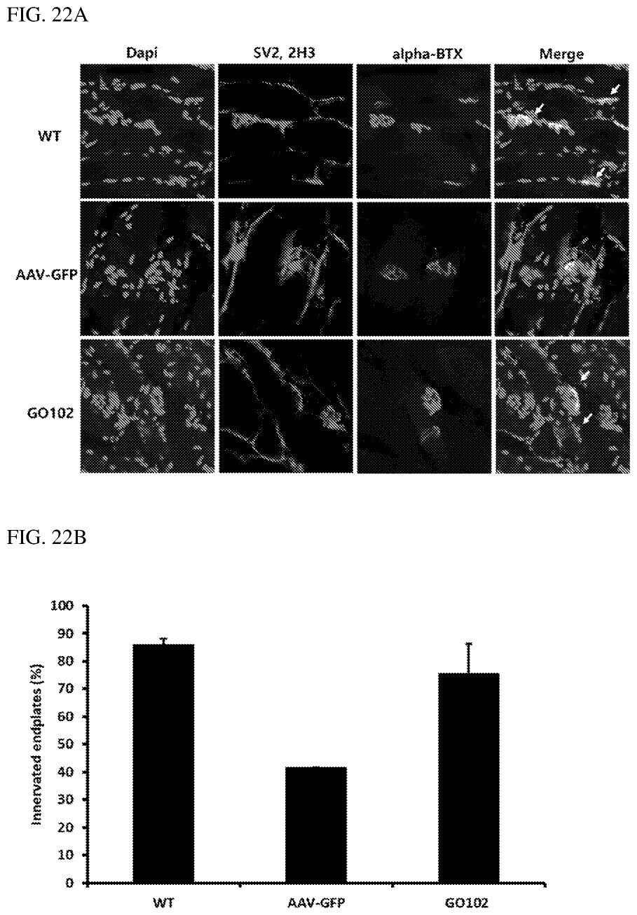

In FIG. 22A, the neuromuscular junctions were stained with alpha-Bungarotoxin, and synaptic vesicle and end plate were staining with SV2 and 2H3. In FIG. 22B, the number of innervated endplates was counted and represented.

GO102 ameliorated the decreased % of innervated endplates (75.6±12.6 vs. 41.0±2.03%) observed in ALS disease model.

Taken together, DX2 inhibits neuromuscular junction (NMJ) damage and it is expected that DX2 restores NMJ block-induced respiratory failure and muscle twitching or fasciculation.

### REFERENCES

KR 10-1067816 (2011).
Rosen D R, et al. (1993) Mutations in Cu/Zn superoxide dismutase gene are associated with familial amyotrophic lateral sclerosis. Nature 362(6415):59-62.
Fridovich I (1995) Superoxide radical and superoxide dismutases. Annu Rev Biochem 64:97-112.
Fridovich I (1997) Superoxide anion radical (O2-·), superoxide dismutases, and related matters. J Biol Chem 272(30):18515-18517.
Bruijn L I, et al. (1998) Aggregation and motor neuron toxicity of an ALS-linked SOD1 mutant independent from wild-type SOD1. Science 281(5384):1851-1854.
Iancu, R, Mohapel, P, Brundin, P, and Paul, G (2005). Behavioral characterization of a unilateral 6-OHDA-lesion model of Parkinson's disease in mice. Behavioural brain research 162: 1-10.
Meredith, G E, and Rademacher, D J (2011). 1VIPTP mouse models of Parkinson's disease: an update. Journal of Parkinson's disease 1: 19-33.
Dawson, T M, and Dawson, V L (2014). Parkin plays a role in sporadic Parkinson's disease. Neuro-degenerative diseases 13: 69-71.
Alzheimer's Association. “2019 Alzheimer's disease facts and figures.” Alzheimer's & Dementia 15.3 (2019): 321-387.
Choi, Jin Woo, et al. “Cancer-associated splicing variant of tumor suppressor AIMP2/p38: pathological implication in tumorigenesis.” PLoS genetics (2011).
Duyckaerts C, Delatour B, Potier M C. “Classification and basic pathology of Alzheimer disease.” Acta Neuropathol. (2009): 5-36.
Viola K L, Klein W L. “Amyloid β oligomers in Alzheimer's disease pathogenesis, treatment, and diagnosis.” Acta Neuropathol. (2015): 183-206.
De Strooper B, Vassar R, Golde T. “The secretases: enzymes with therapeutic potential in Alzheimer disease. ” Nat Rev Neurol. (2010): 99-107.
Finlay C A, Hinds P W, Levine A J. “ The p53 proto-oncogene can act as a suppressor of transformation.” Cell. (1989): 1083-93.
Lee, Y, Karuppagounder, S S, Shin, J H, Lee, Y I, Ko, H S, Swing, D, et al. (2013). Parthanatos mediates AIMP2-activated age-dependent dopaminergic neuronal loss. Nature neuroscience.
Szabo, C, Zingarelli, B, O'Connor, M, and Salzman, A L (1996). DNA strand breakage, activation of poly (ADP-ribose) synthetase, and cellular energy depletion are involved in the cytotoxicity of macrophages and smooth muscle cells exposed to peroxynitrite. Proceedings of the National Academy of Sciences of the United States of America 93: 1753-1758.
Virag, L, Salzman, A L, and Szabo, C (1998). Poly (ADP-ribose) synthetase activation mediates mitochondrial injury during oxidant-induced cell death. Journal of immunology 161: 3753-3759.
Barkauskaite, E, Jankevicius, G, and Ahel, I (2015). Structures and Mechanisms of Enzymes Employed in the Synthesis and Degradation of PARP-Dependent Protein ADP-Ribosylation. Molecular cell 58: 935-946.
Brown et al., Endogenous microRNA regulation suppresses transgene expression in hematopoietic lineages and enables stable gene transfer, Nature Med. 12:585-591 (2006).
Brown et al., Endogenous microRNA can broadly exploited to regulate transggene expression according to tissue, lineage and differentiation state, Nature Biotech. 25:12457-1467 (2007).

The foregoing description of the specific embodiments will so fully reveal the general nature of the invention that others can, by applying knowledge within the skill of the art, readily modify and/or adapt for various applications, without departing from the general concept of the invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

What is claimed is:

1. A method for delaying disease onset of a subject with amyotrophic lateral sclerosis (ALS), comprising administering to the subject a recombinant vector comprising an exon 2-deleted AIMP2 variant (AIMP2-DX2) gene.

2. A method of inhibiting neuronal cell death in a subject with amyotrophic lateral sclerosis (ALS), comprising administering to the subject a recombinant vector comprising an exon 2-deleted AIMP2 variant (AIMP2-DX2) gene.

3. A method of treating muscle atrophy in a subject in need thereof, comprising administering to the subject a recombinant vector comprising an exon 2-deleted AIMP2 variant (AIMP2-DX2) gene. (ALS).

4. The method of claim 4, wherein the subject has amyotrophic lateral sclerosis

5. The method of claim 4, wherein the subject has spinal muscular atrophy (SMA).

6. A method for increasing survival rate or prolonging lifespan of a subject with Parkinson's disease (PD), comprising administering to the subject a recombinant vector comprising an exon 2-deleted AIMP2 variant (AIMP2-DX2) gene.

7. A method of preventing behavior deficit, restoring a motor symptom, and/or reducing neuronal damage in a subject with Parkinson's disease (PD), comprising administering to the subject a recombinant vector comprising an exon 2-deleted AIMP2 variant (AIMP2-DX2) gene.

8. A method of inhibiting amyloid beta oligomer (Aβ-O)-induced neuronal cell death or Aβ-O-induced p53 expression in a subject with Alzheimer's disease (AD), comprising administering to the subject a recombinant vector comprising an exon 2-deleted AIMP2 variant (AIMP2-DX2) gene.

9. A method of inhibiting neuromuscular junction (NMJ) damage in a subject with spinal muscular atrophy (SMA), comprising administering to the subject a recombinant vector comprising an exon 2-deleted AIMP2 variant (AIMP2-DX2) gene.

10. A method of inhibiting neuromuscular junction (NMJ) damage, inhibiting NMJ block induced respiratory failure, difficulty in breathing, inhibiting NMJ block induced muscle twitching or fasciculation, in a subject with amyotrophic lateral sclerosis (ALS), comprising administering to the subject a recombinant vector comprising an exon 2-deleted AIMP2 variant (AIMP2-DX2) gene.

11. A method of suppressing anoikis, and/or increasing laminin receptor stabilization in a subject with amyotrophic lateral sclerosis (ALS), Parkinson's disease (PD), comprising administering to the subject a recombinant vector comprising an exon 2-deleted AIMP2 variant (AIMP2-DX2) gene.

12. The method of any one of claims 1-11, wherein the vector further comprises a miR-142 target sequence.

13. The method of any one of claims 1-12, wherein the vector further comprises a promoter operably linked to the AIMP2-DX2.

14. The method of claim 13, wherein the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter, Synapsin promoter, MeCP2 promoter, CaMKII promoter, Hb9 promoter, or opsin promoter.

15. The method of any one of claims 12-14, wherein the miR-142 target sequence is 3′ to the AIMP2-DX2 gene.

16. The method of any one of claims 1-15, wherein the AIMP2-DX2 gene comprises a nucleotide sequence encoding an amino acid sequence that is at least 90% identical to SEQ ID NO:2, 13, 14, 15, 16, 17, 18, 19, or 20.

17. The method of claim 16, wherein the AIMP2-DX2 gene comprises a nucleotide sequence encoding an amino acid sequence of SEQ ID NO:2, 13, 14, 15, 16, 17, 18, 19, or 20.

18. The method of any one of claims 1-17, wherein the AIMP2-DX2 gene does not have an exon comprising a nucleotide sequence encoding an amino acid sequence that is at least 90% identical to SEQ ID NO:10 or 11.

19. The method of any one of claims 1-18, wherein the AIMP2-DX2 gene does not have an exon comprising a nucleotide sequence encoding an amino acid sequence of SEQ ID NO:10 or 11.

20. The method of any one of claims 12-19, wherein the miR-142 target sequence comprises ACACTA.

21. The method of claim 12-19, wherein the miR-142 target sequence comprises ACACTA and 1-17 additional contiguous nucleotides of SEQ ID NO:5.

22. The method of any one of claims 12-19, wherein the miR-142 target sequence comprises a nucleotide sequence at least 50% identical to a nucleotide sequence of SEQ ID NO:5 (TCCATAAAGTAGGAAACACTACA).

23. The method of claim 22, wherein the miR-142 target sequence comprises a nucleotide sequence of SEQ ID NO:5.

24. The method of any one of claims 12-19, wherein the miR-142 target sequence comprises ACTTTA.

25. The method of claim 12-19, wherein the miR-142 target sequence comprises ACTTTA and 1-15 additional contiguous nucleotides of SEQ ID NO:7.

26. The method of any one of claims 12-19, wherein the miR-142 target sequence comprises a nucleotide sequence at least 50% identical to a nucleotide sequence of SEQ ID NO:7 (AGTAGTGCTTTCTACTTTATG).

27. The method of claim 26, wherein the miR-142 target sequence comprises a nucleotide sequence of SEQ ID NO:7.

28. The method of any one of claims 12-27, wherein the miR-142 target sequence is repeated 2-10 times.

29. The method of any one of claims 1-28, wherein the vector is a viral vector.

30. The method of claim 29, wherein the viral vector is an adenovirus, adeno-associated virus, lentivirus, retrovirus, human immunodeficiency virus (HIV), murine leukemia virus (MLU), avian sarcoma/leukosis (ASLV), spleen necrosis virus (SNV), Rous sarcoma virus (RSV), mouse mammary tumor virus (MMTV), vaccinia virus, or Herpes simplex virus vector.


SEQUENCE LISTING (25 sequences)

SEQ ID NO: 1 - DNA, 756 bp, Artificial Sequence
  AIMP2-DX2
atgccgatgtaccaggtaaagccctatcacgggggcggcgcgcctctccgtgtggagcttcccacctgcatgtaccggctccccaacgtgcacggcaggagctacggcccagcgccgggcgctggccacgtgcaggattacggggcgctgaaagacatcgtgatcaacgcaaacccggcctcccctcccctctccctgcttgtgctgcacaggctgctctgtgagcacttcagggtcctgtccacggtgcacacgcactcctcggtcaagagcgtgcctgaaaaccttctcaagtgctttggagaacagaataaaaaacagccccgccaagactatcagctgggattcactttaatttggaagaatgtgccgaagacgcagatgaaattcagcatccagacgatgtgccccatcgaaggcgaagggaacattgcacgtttcttgttctctctgtttggccagaagcataatgctgtcaacgcaacccttatagatagctgggtagatattgcgatttttcagttaaaagagggaagcagtaaagaaaaagccgctgttttccgctccatgaactctgctcttgggaagagcccttggctcgctgggaatgaactcaccgtagcagacgtggtgctgtggtctgtactccagcagatcggaggctgcagtgtgacagtgccagccaatgtgcagaggtggatgaggtcttgtgaaaacctggctccttttaacacggccctcaagctccttaagtga

SEQ ID NO: 2 - protein, 251 aa, Artificial Sequence
  AIMP2-DX2
MPMYQVKPYHGGGAPLRVELPTCMYRLPNVHGRSYGPAPGAGHVQDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 3 - DNA, 936 bp, Artificial Sequence
  AIMP2
atgccgatgtaccaggtaaagccctatcacgggggcggcgcgcctctccgtgtggagcttcccacctgcatgtaccggctccccaacgtgcacggcaggagctacggcccagcgccgggcgctggccacgtgcaggaagagtctaacctgtctctgcaagctcttgagtcccgccaagatgatattttaaaacgtctgtatgagttgaaagctgcagttgatggcctctccaagatgattcaaacaccagatgcagacttggatgtaaccaacataatccaagcggatgagcccacgactttaaccaccaatgcgctggacttgaattcagtgcttgggaaggattacggggcgctgaaagacatcgtgatcaacgcaaacccggcctcccctcccctctccctgcttgtgctgcacaggctgctctgtgagcacttcagggtcctgtccacggtgcacacgcactcctcggtcaagagcgtgcctgaaaaccttctcaagtgctttggagaacagaataaaaaacagccccgccaagactatcagctgggattcactttaatttggaagaatgtgccgaagacgcagatgaaattcagcatccagacgatgtgccccatcgaaggcgaagggaacattgcacgtttcttgttctctctgtttggccagaagcataatgctgtcaacgcaacccttatagatagctgggtagatattgcgatttttcagttaaaagagggaagcagtaaagaaaaagccgctgttttccgctccatgaactctgctcttgggaagagcccttggctcgctgggaatgaactcaccgtagcagacgtggtgctgtggtctgtactccagcagatcggaggctgcagtgtgacagtgccagccaatgtgcagaggtggatgaggtcttgtgaaaacctggctcctttt

SEQ ID NO: 4 - DNA, 207 bp, Artificial Sequence
  Exon 2 of AIMP2
gaagagtctaacctgtctctgcaagctcttgagtcccgccaagatgatattttaaaacgtctgtatgagttgaaagctgcagttgatggcctctccaagatgattcaaacaccagatgcagacttggatgtaaccaacataatccaagcggatgagcccacgactttaaccaccaatgcgctggacttgaattcagtgcttgggaag

SEQ ID NO: 5 - DNA, 23 bp, Artificial Sequence
  miR-142-3p target sequence
tccataaagtaggaaacactaca

SEQ ID NO: 6 - DNA, 132 bp, Artificial Sequence
  4 repeats of miR-142-3p target sequence
ccagaagcttgctagctccataaagtaggaaacactacatcactccataaagtaggaaacactacagatatctccataaagtaggaaacactacatcactccataaagtaggaaacactacaaagcttgtag

SEQ ID NO: 7 - DNA, 21 bp, Artificial Sequence
  miR-142-5p target sequence
agtagtgctttctactttatg

SEQ ID NO: 8 - DNA, 24 bp, Artificial Sequence
  AIMP2 variant forward primer
ctggccacgtgcaggattacgggg

SEQ ID NO: 9 - DNA, 20 bp, Artificial Sequence
  AIMP2 variant reverse primer
aagtgaatcccagctgatag

SEQ ID NO: 10 - protein, 69 aa, Artificial Sequence
  Exon 2 of AIMP2
EESNLSLQALESRQDDILKRLYELKAAVDGLSKMIQTPDADLDVTNIIQADEPTTLTTNALDLNSVLGK

SEQ ID NO: 11 - protein, 36 aa, Artificial Sequence
  Exon 2 of AIMP2 variant
MIQTPDADLDVTNIIQADEPTTLTTNALDLNSVLGK

SEQ ID NO: 12 - protein, 320 aa, Artificial Sequence [sequence omitted]

SEQ ID NO: 13 - protein, 243 aa, Artificial Sequence
  AIMP-DX2 variant 1
MPMYQVKPYHGGGAPLRVELPTCMYRLPNVHGRSYGPAPGAGHVQDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPF

SEQ ID NO: 14 - protein, 244 aa, Artificial Sequence
  AIMP2-DX2 variant 2
MQMEGTAHVKICGQSQGGGLGTPRTVWLEHRQRTKLGEDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 15 - protein, 207 aa, Artificial Sequence
  AIMP2-DX2 variant 3
MDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 16 - protein, 222 aa, Artificial Sequence
  AIMP2-DX2 variant 4
MNSPAVNTLIQRSRHGDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 17 - protein, 211 aa, Artificial Sequence
  AIMP2-DX2 variant 5
MPMYQDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 18 - protein, 251 aa, Artificial Sequence; identical to SEQ ID NO: 2
  AIMP2-DX2 variant 6

SEQ ID NO: 19 - protein, 206 aa, Artificial Sequence
  AIMP2-DX2 variant 7
DYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 20 - protein, 198 aa, Artificial Sequence
  AIMP2-DX2 consensus sequence
DYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPF

SEQ ID NO: 21 - DNA, 16 bp, Artificial Sequence
  Nhe1, Hind III, and Bmt1 sites in miR-142-3pT with 4 repeats
ccagaagcttgctagc

SEQ ID NO: 22 - DNA, 10 bp, Artificial Sequence
  Hind H site in miR-142-3pT with 4 repeats
aagcttgtag

SEQ ID NO: 23 - RNA, 23 nt, Artificial Sequence
  miR-142-3p
uguaguguuuccuacuuuaugga

SEQ ID NO: 24 - RNA, 21 nt, Artificial Sequence
  miR-142-5p
cauaaaguagaaagcacuacu

SEQ ID NO: 25 - DNA, 238 bp, Artificial Sequence
  miR-142-3pT target sequence mutant
ccgctgcagtgtgacagtgccagccaatgtgcagaggtggatgaggtcttgtgaaaacctggctccttttaacacggccctcaagctccttaagtgaccagaagcttgctagctccataaagtaggaccactgcaatcactccataaagtaggaccactgcaagatatctccataaagtaggaccactgcaatcactccataaagtaggaccactgcaaaagcttgtagggatccgcc